(19) United States
(12) Patent Application Publication
(10) Pub. No.: US 2025/0169682 A1
(43) Pub. Date: May 29, 2025

(54) OPERATION SECTION AND INSERTION DEVICE

(71) Applicant: OLYMPUS MEDICAL SYSTEMS CORP., Tokyo (JP)

(72) Inventor: Takumi KAWAKAMI, Tokyo (JP)

(73) Assignee: OLYMPUS MEDICAL SYSTEMS CORP., Tokyo (JP)

(21) Appl. No.: 18/948,666

(22) Filed: Nov. 15, 2024

Related U.S. Application Data
(60) Provisional application No. 63/602,461, filed on Nov. 24, 2023.

(51) Int. Cl. A61B 1/005

(52) U.S. Cl. CPC A61B 1/0052; A61B 1/0057

(57) ABSTRACT

An operation section for use with an endoscope comprises a shaft, a wire fixing surface connected to the shaft, a first to a third wires having a fixing part attached to the wire fixing surface, and a first wire guide configured to guide the first wire, a second wire guide configured to guide the second wire, and a third wire guide configured to guide the third wire. The first wire includes a first portion between the fixing part of the first wire and the first wire guide, and the second wire includes a second portion between the fixing part of the second wire and the second wire guide. In a view from a neutral axis of the shaft, the first and second portions cross.

### RELATED APPLICATION DATA

This application is based on and claims priority under 35 U.S.C. § 119 to U.S. Provisional Application No. 63/602,461, filed on Nov. 24, 2023, the entire contents of which are incorporated herein by reference.

### FIELD OF DISCLOSURE

The disclosure is a disclosure related to an insertion device, such as an endoscope or the like, including a bending operation mechanism for achieving a bending motion of a bending portion in conjunction with a tilting operation on a bending operation lever.

### BACKGROUND

Conventionally, insertion devices such as endoscopes and the like have been widely used in medical and industrial fields, for example. An endoscope or the like as an example of the insertion device includes an insertion section that has flexibility and that is formed into an elongated tubular shape, and an operation section that is continuously provided on a proximal end side of the insertion section and that includes various operation members and the like on an outer surface. Furthermore, a bending portion that is formed to be bendable is provided at a partial region close to a distal end of the insertion section. The bending portion has a configuration for achieving active bending in up, down, left and right directions around an insertion axis of the insertion section in response to an operation input on a predetermined operation member provided at the operation section. In this manner, conventional endoscopes and the like include a bending operation mechanism of a predetermined mode inside the operation section.

With respect to a conventional endoscope or the like, a joystick-type bending operation lever is well known as an operation member for bending the bending portion, for example. The joystick-type bending operation lever is generally capable of tilting in any direction including the up, down, left and right directions.

By adopting a joystick-type bending operation lever of such a mode, the bending operation mechanism of a conventional endoscope or the like may, with a simpler configuration, achieve a bending motion of the bending portion in a predetermined range in any direction including the up, down, left and right directions.

Furthermore, the bending operation mechanism of an endoscope or the like adopts a pull mechanism that uses a plurality of wire members as a mechanism for achieving a bending motion in the up, down, left and right directions, for example. Normally, a mechanism including four wire members is generally used in relation to the bending motion of the bending portion in the up, down, left and right directions.

In the pull mechanism of a conventional endoscope or the like, a plurality of wire members are disposed by being inserted through the insertion section from a proximal end side of the insertion section (inside the operation section) to a distal end side of the insertion section (a distal end of the bending portion).

Generally, with an insertion device such as an endoscope, there is a constant demand to reduce a diameter of the insertion section. To reduce the diameter of the insertion section, it is effective to reduce structural members disposed inside the insertion section as much as possible.

Accordingly, in recent years, as a configuration that may achieve reduction in the diameter of the insertion section while achieving bending motion of the bending portion in any of the up, down, left and right directions, various insertion devices, such as endoscopes and the like, that include three wire members have been proposed by U.S. Patent Application Publication No. 2023/0219212 A1 and the like.

The insertion devices disclosed in U.S. Patent Application Publication No. 2023/0219212 A1 and the like include a bending operation mechanism including a joystick-type bending operation lever and three wire members. The bending operation mechanism has a configuration where any one of the three wire members is pulled in conjunction with a tilting operation on the joystick-type bending operation lever, and may thus cause the bending portion to bend in a predetermined direction.

### SUMMARY

An operation section for use with an endoscope comprises a shaft, a wire fixing surface connected to the shaft, a first to a third wires having a fixing part attached to the wire fixing surface, and a first wire guide configured to guide the first wire, a second wire guide configured to guide the second wire, and a third wire guide configured to guide the third wire. The first wire includes a first portion between the fixing part of the first wire and the first wire guide, and the second wire includes a second portion between the fixing part of the second wire and the second wire guide. In a view from a neutral axis of the shaft, the first and second portions cross.

An insertion device of a mode of the present disclosure includes an operation section; a bending portion provided at an insertion section provided continuously to the operation section; first to third wires each having a distal end attached to the bending portion, where, when a proximal end is pulled, each wire causes the bending portion to bend in a predetermined direction; a lever including a shaft member that extends from inside of the operation section to outside of the operation section, the lever being tilted under an operation force from the outside of the operation section; a wire fixing member configured to allow each of the proximal ends of the first to third wires to be fixed at mutually different positions inside the operation section, and to pull the first to third wires in conjunction with tilting of the lever; and first to third wire guides configured to guide the first to third wires, respectively, at mutually different positions in a state where tension is applied, and to separately change each of wire pulling directions, where each of the proximal ends of the first to third wires is fixed to the wire fixing member while being disposed in mutually different quadrants on an XY-plane that is a plane set by an X-axis orthogonal to the shaft member and a Y-axis orthogonal to the X-axis, the XY-plane having a point of origin at an intersection point of the X-axis, the Y-axis, and the shaft member, the first wire guide guides the first wire to which tension is applied, at a position, on the XY-plane, opposite to the proximal end of the first wire across the X-axis, the second wire guide guides the second wire to which tension is applied, at a position, on the XY-plane, opposite to the proximal end of the second wire across the X-axis and the Y-axis, and the third wire guide guides the third wire to which tension is applied, at a position, on the XY-plane, opposite to the proximal end of the third wire across the X-axis and the Y-axis.

### BRIEF DESCRIPTION OF THE DRAWINGS

FIG. 1 is an external view showing a schematic configuration of an endoscope as an example of an insertion device of an embodiment of the present disclosure.

FIG. 2 is a main part enlarged perspective view showing a bending tube forming a main part of a bending portion of the endoscope in FIG. 1.

FIG. 3 is a main part enlarged perspective view showing an internal configuration of an operation section, structural members related to a bending operation mechanism, where a second outer shell of the endoscope in FIG. 1 is removed.

FIG. 4 is an exploded perspective view showing a part of a first outer shell and a part of the bending operation mechanism of the endoscope in FIG. 1.

FIG. 5 is an exploded perspective view showing a part of the second outer shell and a part of the bending operation mechanism of the endoscope in FIG. 1.

FIG. 6 is a main part enlarged perspective view showing a bending operation unit of the endoscope in FIG. 1.

FIG. 7 is a cross-sectional view taken along a line [VII]-[VII] in FIG. 1.

FIG. 8 is a vertical cross-sectional view seen in an arrow [8] direction in FIG. 1, and is a diagram showing a state where a bending operation lever is at a neutral position.

FIG. 9 is an explanatory diagram showing a relationship between a recessed surface and a restriction member at the bending operation lever included in the bending operation mechanism of the endoscope, and is a diagram showing a state where the bending operation lever is at the neutral position.

FIG. 10 is a vertical cross-sectional view seen in the arrow [8] direction in FIG. 1, and is a diagram showing a state where the bending operation lever is tilted downward (arrow D direction).

FIG. 11 is an explanatory diagram showing a relationship between the recessed surface and the restriction member at the bending operation lever included in the bending operation mechanism of the endoscope, and is a diagram showing a state where the bending operation lever is tilted downward (arrow D direction).

FIG. 12 is a vertical cross-sectional view showing, from a side of the operation section, a state of the bending operation unit and a plurality of bending wires when the bending operation lever of the endoscope in FIG. 1 is at the neutral position.

FIG. 13 is a conceptual diagram showing, from a front of the operation section (arrow [13] direction in FIG. 12), the state of the bending operation unit and the plurality of bending wires when the bending operation lever of the endoscope in FIG. 1 is at the neutral position.

FIG. 14 is a vertical cross-sectional view showing, from the side of the operation section, a state of the bending operation unit and the plurality of bending wires when the bending operation lever of the endoscope in FIG. 1 is tilted in an upward direction (arrow U direction).

FIG. 15 is a conceptual diagram showing, from the front of the operation section, the bending operation unit and the plurality of bending wires in the state shown in FIG. 14.

FIG. 16 is a vertical cross-sectional view showing, from the side of the operation section, a state of the bending operation unit and the plurality of bending wires when the bending operation lever of the endoscope in FIG. 1 is tilted in a downward direction (arrow D direction).

FIG. 17 is a conceptual diagram showing, from the front of the operation section, the bending operation unit and the plurality of bending wires in the state shown in FIG. 16.

FIG. 18 is a diagram showing, in relation to the endoscope in FIG. 1, a state of the bending operation unit and the plurality of bending wires when the bending operation lever is tilted in a right direction (arrow R direction).

FIG. 19 is a diagram showing, in relation to the endoscope in FIG. 1, a state of the bending operation unit and the plurality of bending wires when the bending operation lever is tilted in a left direction (arrow L direction).

FIG. 20 is a diagram showing a first modification of the endoscope of the embodiment of the present disclosure.

FIG. 21 is a diagram showing a second modification of the endoscope of the embodiment of the present disclosure.

FIG. 22 is a diagram showing a third modification of the endoscope of the embodiment of the present disclosure.

FIG. 23 is a diagram showing a fourth modification of the endoscope of the embodiment of the present disclosure.

FIG. 24 is a diagram showing a fifth modification of the endoscope of the embodiment of the present disclosure, and is a main part enlarged perspective view showing only a wire guide pin.

FIG. 25 is a diagram showing an assembled state, inside the operation section, of the wire guide pin in FIG. 24.

FIG. 26 is a diagram showing an example arrangement, inside the operation section, of the wire guide pin in FIG. 24.

FIG. 27 is a diagram showing a sixth modification of the endoscope of the embodiment of the present disclosure.

FIG. 28 is a diagram showing a seventh modification of the endoscope of the embodiment of the present disclosure.

FIG. 29 is a diagram showing an eighth modification of the endoscope of the embodiment of the present disclosure.

FIG. 30 is a diagram showing a ninth modification of the endoscope of the embodiment of the present disclosure.

FIG. 31 is a diagram showing a tenth modification of the endoscope of the embodiment of the present disclosure, and is a perspective view of the bending operation unit.

FIG. 32 is a side cross-sectional view of the bending operation unit in FIG. 31.

FIG. 33 is a diagram showing an eleventh modification of the endoscope of the embodiment of the present disclosure, and is a perspective view of the bending operation unit.

FIG. 34 is a side view of the bending operation unit in FIG. 33.

FIG. 35 is a diagram showing a twelfth modification of the endoscope of the embodiment of the present disclosure.

FIG. 36 is a diagram showing a thirteenth modification of the endoscope of the embodiment of the present disclosure.

FIG. 37 is a diagram showing a fourteenth modification of the endoscope of the embodiment of the present disclosure.

FIG. 38 is a diagram showing a fifteenth modification of the endoscope of the embodiment of the present disclosure.

### DETAILED DESCRIPTION

Generally, with an insertion device of a conventional mode as disclosed in U.S. Patent Application Publication No. 2023/0219212 A1 or the like, when a tilting operation is performed on a joystick-type bending operation lever to achieve a predetermined bending motion, if a predetermined tilting operation range is exceeded, an unintended wire member among three wire members may possibly be pulled. In such a case, there is a problem that an unstable bending motion, such as a bending motion of a bending portion in an unintended direction different from a predetermined bending direction, is performed.

The present disclosure can provide an insertion device, such as an endoscope, including a bending operation mechanism for achieving a bending motion of a bending portion by pulling any one of a plurality of (at least three) wire members in conjunction with a tilting operation on a joystick-type bending operation lever, where the insertion device is capable of constantly achieving a stable bending motion within a predetermined tilting range of the joystick-type bending operation lever.

Hereinafter, the present disclosure will be described using an embodiment shown in the drawings. Each diagram used in the description below is schematic. In each diagram, each structural component is shown large enough to be recognized in the diagram. Accordingly, a dimensional relationship, scales and the like of members in the diagrams may be shown differently for each structural component. Accordingly, the present disclosure is not limited to the mode shown in the drawings with respect to the number of pieces of each structural component, the shape of each structural component, a ratio of respective sizes of structural components, relative positional relationships of respective structural components, and the like shown in each drawing.

First, a configuration of an insertion device of an embodiment of the present disclosure will be described below.

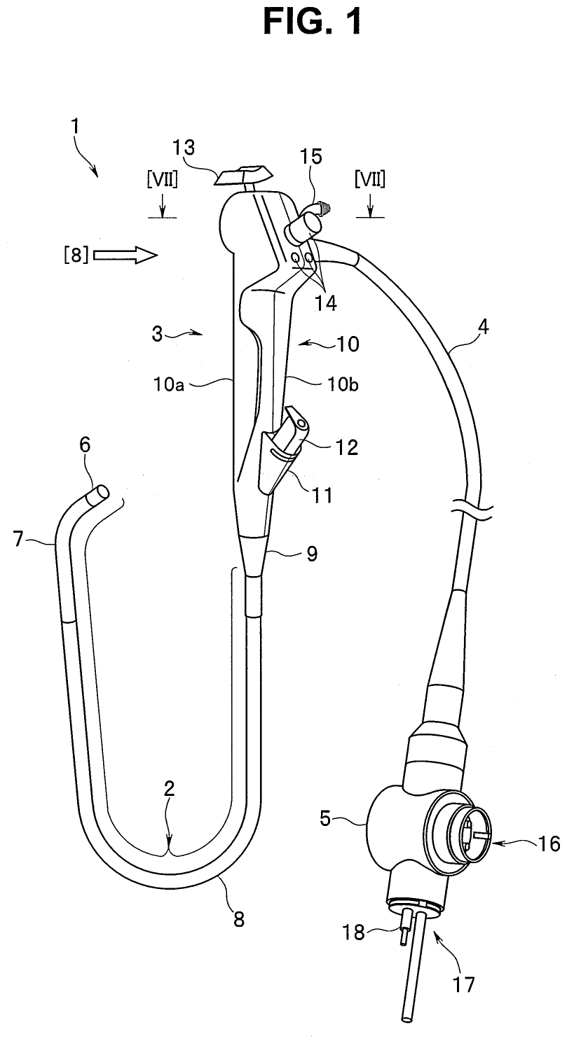

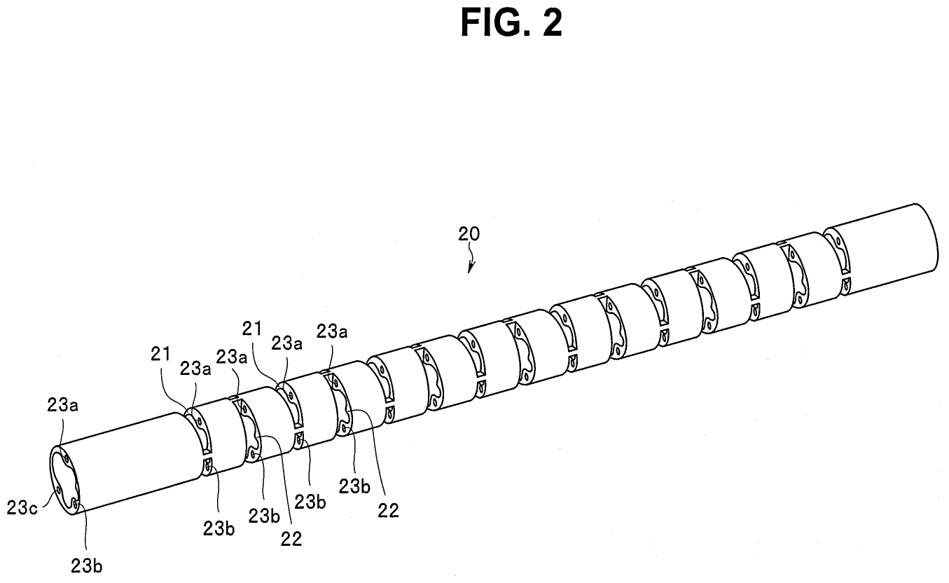

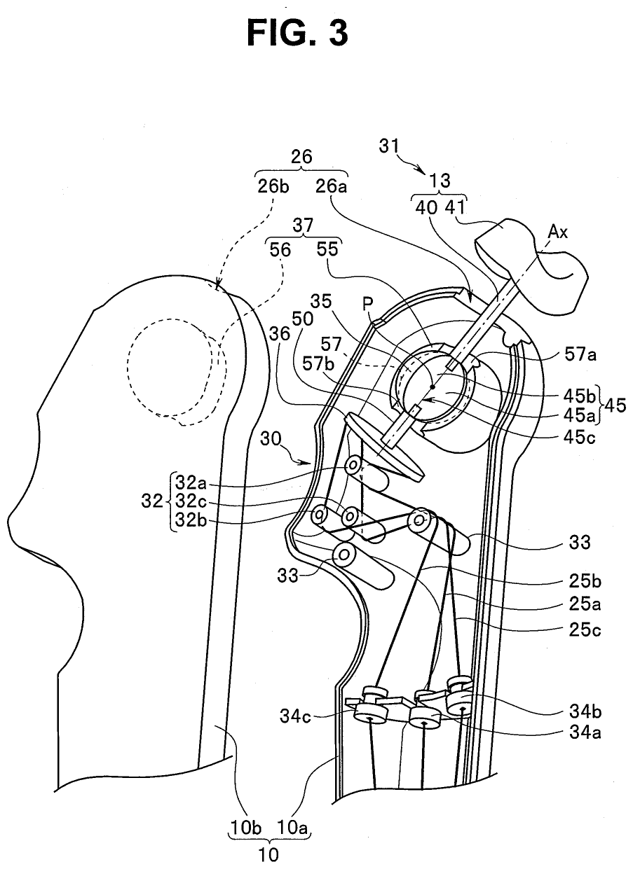

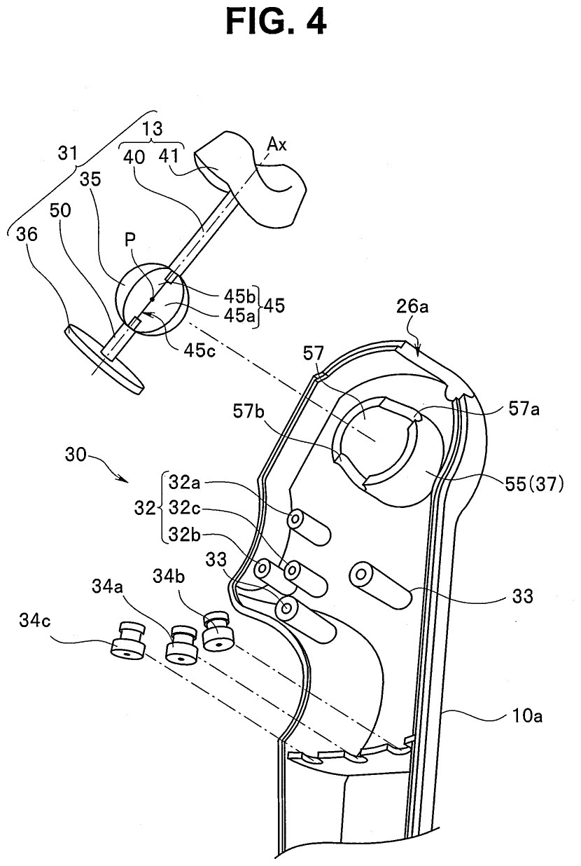

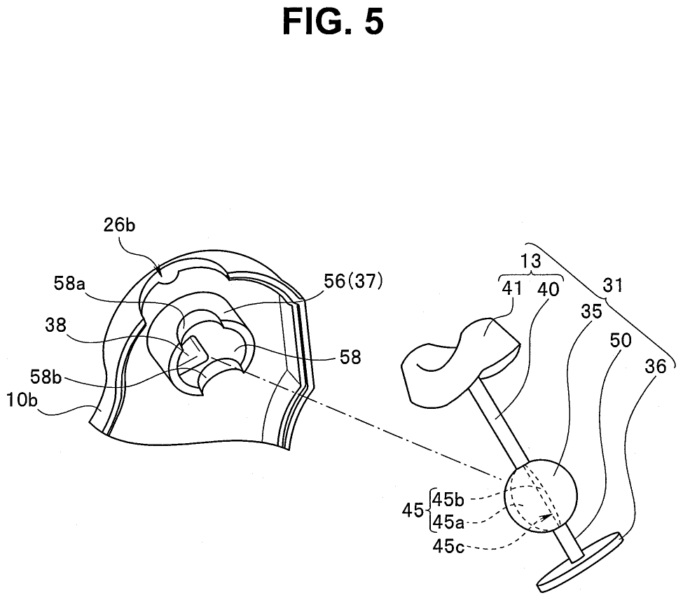

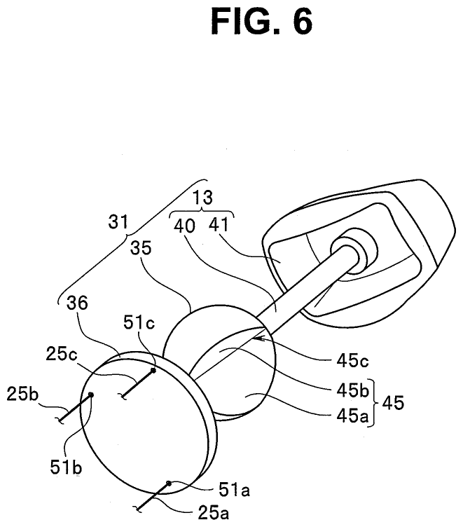

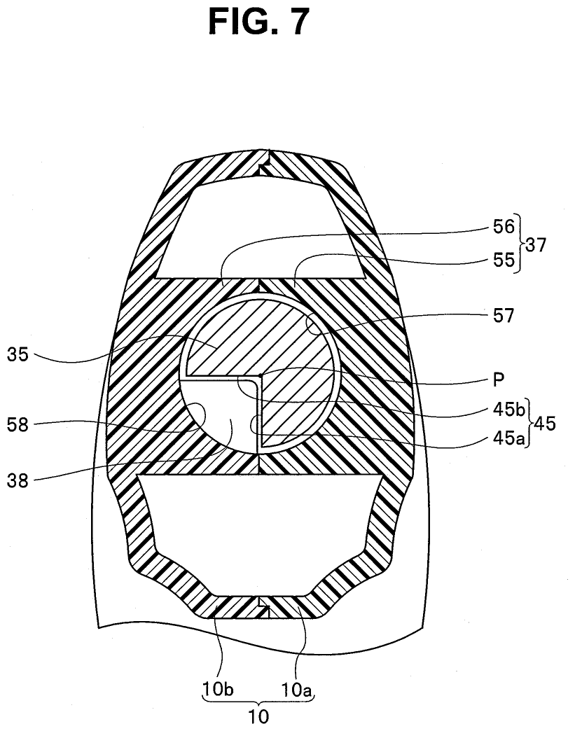

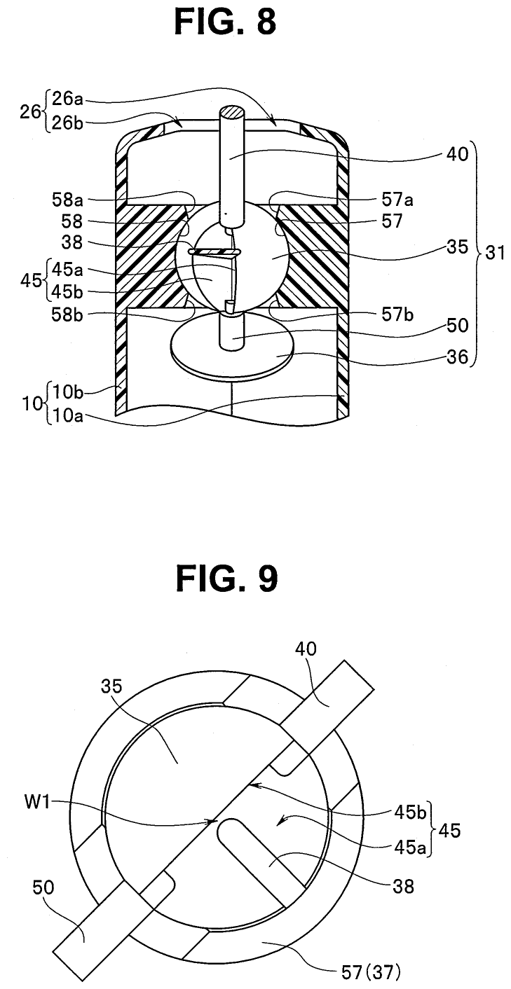

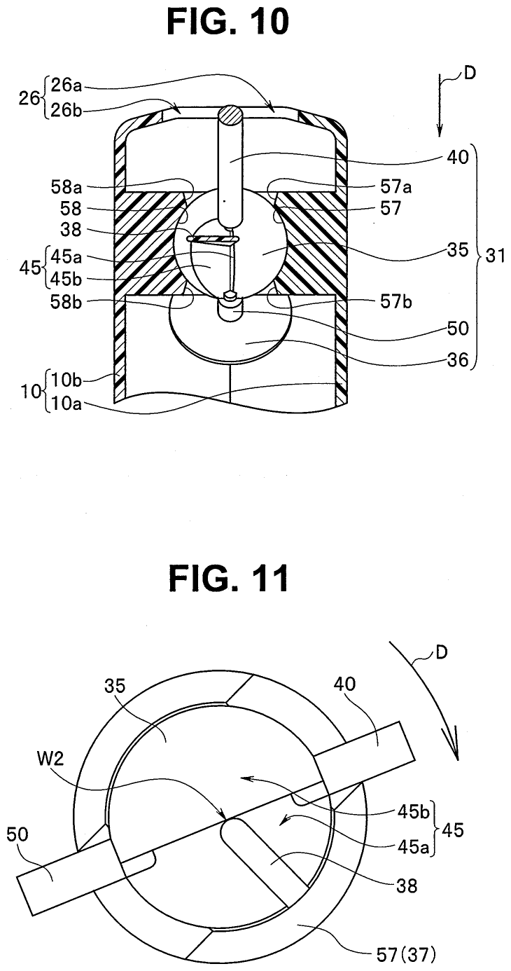

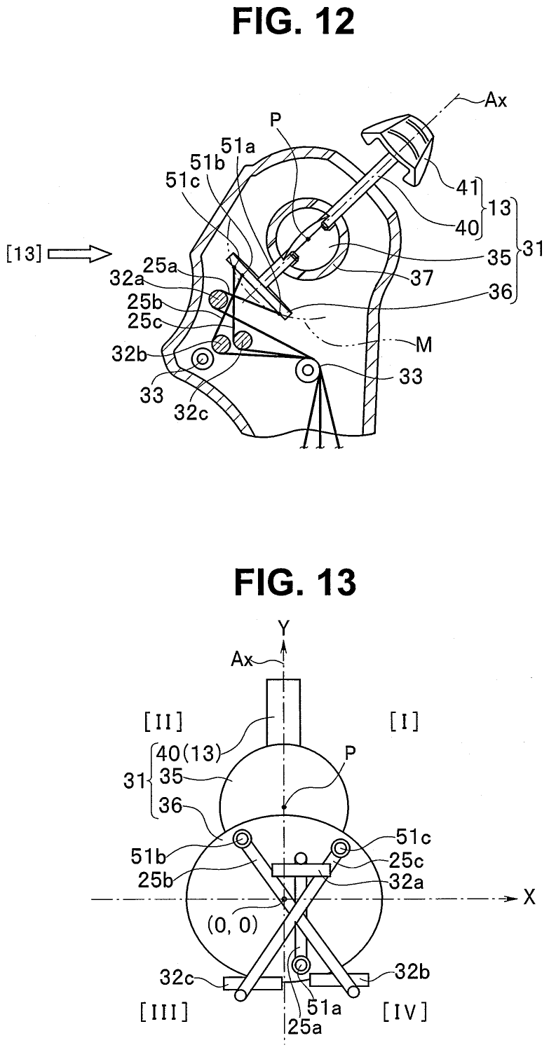

FIG. 1 is an external view showing a schematic configuration of an endoscope as an example of an insertion device of an embodiment of the present disclosure. FIG. 2 is a main part enlarged perspective view showing a bending tube forming a main part of a bending portion of the endoscope in FIG. 1. FIG. 3 is a main part enlarged perspective view showing an internal configuration of an operation section, structural members related to a bending operation mechanism, where a second outer shell of the endoscope in FIG. 1 is removed. FIG. 4 is an exploded perspective view showing a part of a first outer shell and a part of the bending operation mechanism of the endoscope in FIG. 1. FIG. 5 is an exploded perspective view showing a part of the second outer shell and a part of the bending operation mechanism of the endoscope in FIG. 1. FIG. 6 is a main part enlarged perspective view showing a bending operation unit of the endoscope in FIG. 1. FIG. 7 is a cross-sectional view taken along a line [VII]-[VII] in FIG. 1. FIGS. 8 and 10 are vertical cross-sectional views seen in an arrow [8] direction in FIG. 1. Of the drawings, FIG. 8 shows a state where a bending operation lever is at a neutral position. FIG. 10 shows a state where the bending operation lever is tilted downward (arrow D direction). FIGS. 9 and 11 are explanatory diagrams showing a relationship between a recessed surface and a restriction member at the bending operation lever included in the bending operation mechanism of the endoscope in FIG. 1. Of the drawings, FIG. 9 shows a state where the bending operation lever is at the neutral position. FIG. 11 shows a state where the bending operation lever is tilted downward (arrow D direction). FIGS. 12 and 13 are diagrams showing a state of the bending operation unit and a plurality of bending wires when the bending operation lever is at the neutral position. Of the drawings, FIG. 12 is a vertical cross-sectional view showing the state of the bending operation unit and the plurality of bending wires when the operation section is seen from a side. Additionally, FIG. 12 shows only the outer shell in cross-section. FIG. 13 is a diagram conceptually showing the state of the bending operation unit and the plurality of bending wires seen from a front of the operation section (arrow [13] direction in FIG. 12). The first to the third wire guides (32a, 32b, 32c) can be provided on an opposite side of the shaft (40, 50) with respect to the wire fixing surface 36.

For example, an endoscope of the present embodiment is an endoscope for urinary bladder/renal pelvis/urinary organs. Furthermore, the endoscope of the present embodiment is assumed to be a single-use endoscope that is disposed of after being inserted in a subject for a single examination, for example.

A single-use endoscope is disposed after being used once, and should not be used multiple times. The single-use endoscope can include a single-use component and a multi-use component. The single-use component is disposed or returned to manufacturer of after being used once, and the multi-use component is a reusable component that is repeatedly used. Even the reusable endoscope capable of being used multiple times by being reprocessed can be stopped being used and be sent to the manufacturer or the like for maintenance after being used a predetermined number of times.

As shown in FIG. 1, an endoscope 1 includes an insertion section 2, an operation section 3, a universal cord 4, an endoscope connector 5, and the like.

The insertion section 2 is a tubular member that is configured to be inserted inside a subject. As a whole, the insertion section 2 is formed to have an elongated tubular shape having flexibility. The insertion section 2 includes a distal end portion 6, a bending portion 7, and a flexible tube portion 8 that are continuously provided in a stated order from a distal end side.

The distal end portion 6 includes an image pickup unit (not shown), which is an image pickup apparatus including, on inside, an image pickup device and the like, and an illumination unit (not shown) or the like for radiating illumination light toward front side.

Note that a mode of the endoscope 1 to which the present disclosure is applicable is not limited to the example described above (an electronic endoscope including an image pickup unit and the like). For example, another mode of the endoscope 1 may be a so-called fiber scope where the image pickup unit is not included but an image guide fiber or the like is disposed in the insertion section 2.

The bending portion 7 is configured to be capable of actively bending in response to a tilting operation on a bending operation lever 13 among operation members provided at the operation section 3. More specifically, the bending portion 7 is configured to be capable of bending in any direction, around an insertion axis of the insertion section 2, including four directions of up and down directions and left and right directions that intersect the up and down directions. The up and down directions and the left and right directions of the endoscope 1 of the present embodiment are defined in association with up and down directions and left and right directions in an image (not shown) that is picked up by the image pickup unit.

As a component for achieving the bending motion in all of the up, down, left and right directions, the endoscope 1 includes a bending tube 20 (see FIG. 2) inside the insertion section 2, at a position close to a distal end, the bending tube 20 forming a main part of the bending portion 7. A configuration of the bending tube 20 will be briefly described with reference to FIG. 2.

As shown in FIG. 2, the bending tube 20 is a tubular member that is, as a whole, formed to have an elongated tubular shape where first slits 21 that form a pair in the up-down direction and second slits 22 that form a pair in the left-right direction are formed, the first slits 21 and the second slits 22 being alternately formed in plurality in a longitudinal axis direction.

A plurality of wire insertion holes are formed in the bending tube 20 in the longitudinal axis direction. In the example shown in FIG. 2, three wire insertion holes, that is, a first wire insertion hole 23a, a second wire insertion hole 23b, and a third wire insertion hole 23c, are formed as the plurality of wire insertion holes, for example. Bending wires as a plurality of puller wires described later are inserted through the wire insertion holes (23a, 23b, 23c), respectively.

In the example of the present embodiment, as the plurality of bending wires, at least three wire members are provided, the three wire members being a first bending wire 25a as a first wire, a second bending wire 25b as a second wire, and a third bending wire 25c as a third wire (details will be given later; see FIG. 3 and the like).

The three bending wires (25a, 25b, 25c) are inserted through the three wire insertion holes (23a, 23b, 23c), respectively. In the present case, the three bending wires (25a, 25b, 25c) are disposed inside the three wire insertion holes (23a, 23b, 23c), respectively, in a manner capable of moving forward and backward in the longitudinal axis direction.

Furthermore, a distal end of each of the three bending wires (25a, 25b, 25c) is fixed at a predetermined position on a distal end member of the bending tube 20. A proximal end of each of the three bending wires (25a, 25b, 25c) is fixed at a predetermined position on a wire fixing member (wire fixing surface) 36 of a bending operation unit 31 (a unit including the bending operation lever 13) described later (details will be given later; see FIG. 3 and the like).

According to such a configuration, when a tilting operation is performed on the bending operation unit 31 (the bending operation lever 13), and one or two bending wires among the three bending wires are pulled and a remaining bending wire(s) is loosened, the bending portion 7 including the bending tube 20 is bent by a predetermined amount in a predetermined direction according to amounts of pull on the three bending wires.

Referring back to FIG. 1, the flexible tube portion 8 is a soft tubular member that is capable of being passively flexed. A treatment instrument insertion channel, and various electrical signal lines, a light guide for guiding illumination light, and the like (none of which are shown) extending from the image pickup unit are inserted inside the flexible tube portion 8.

The operation section 3 is continuously provided on a proximal end side of the insertion section 2. A bend preventing portion 9 is provided at a connection site between the operation section 3 and a proximal end of the insertion section 2. The bend preventing portion 9 is a structural member that prevents excessive bending of the flexible insertion section 2.

An outer shell 10 forming a housing of the operation section 3 includes a first outer shell 10a and a second outer shell 10b. In the present embodiment, the first outer shell 10a and the second outer shell 10b are separated into left and right relative to the longitudinal axis direction of the operation section 3. The first outer shell 10a and the second outer shell 10b are joined together to form an outer surface of the operation section 3.

Furthermore, the operation section 3 includes a treatment instrument insertion portion 11, the bending operation lever 13, a plurality of operation buttons 14, and a suction valve 15, and the like, for example.

The treatment instrument insertion portion 11 includes a treatment instrument insertion opening (not shown) for allowing insertion of various treatment instruments (not shown). Furthermore, the treatment instrument insertion portion 11 communicates with the treatment instrument insertion channel at a position inside the operation section 3. Note that a forceps plug 12 for blocking the treatment instrument insertion opening is removably attached to the treatment instrument insertion portion 11.

The bending operation lever 13 is a lever member that includes a shaft-shaped member that is disposed protruding from the outer surface of the operation section 3. The bending operation lever 13 is a so-called joystick-type bending operation lever that is supported inside the operation section 3 so as to be capable of tilting in any direction around an axis of the shaft-shaped member.

The bending operation lever 13 is an operation member forming a part of a bending operation mechanism 30 (see FIG. 3 and the like) described later. Although details will be given later, the bending operation mechanism 30 selectively pulls or loosens the plurality of bending wires (25a, 25b, 25c) according to a tilted state of the bending operation lever 13. A configuration is thereby implemented where, when the bending operation lever 13 is tilted, the bending portion 7 can be bent in a predetermined direction by a predetermined amount.

Various functions of the endoscope 1 can be assigned to the plurality of operation buttons 14. Various functions that can be assigned to the operation buttons 14 include functions for performing a gas/liquid feeding operation and a suction operation, and various functions for operating the image pickup unit, the illumination unit and the like, for example.

The suction valve 15 is a connection part for connecting a suction channel to a suction device, not shown.

The universal cord 4 is a hollow tubular member that has flexibility and that extends from the operation section 3. Various signal lines, the light guide, a gas/liquid feeding tube, and the like that are inserted through the insertion section 2 to extend through the inside of the operation section 3 are inserted through the universal cord 4.

The endoscope connector 5 is disposed on an extension end part of the universal cord 4. An electrical connector section 16 is provided on a side surface portion of the endoscope connector 5, for example. The electrical connector section 16 is a connection member that electrically connects a signal line inserted through the universal cord 4 to a processor (not shown), which is an external device.

Furthermore, a light source connector section 17 and a gas/liquid feeding plug 18 are provided on a distal end surface of the endoscope connector 5. The light source connector section 17 is a connection member that optically connects the light guide inserted through the universal cord 4 to a light source device (not shown), which is an external device. The gas/liquid feeding plug 18 is a connection member that connects the gas/liquid feeding tube inserted through the universal cord to a gas/liquid feeding device (not shown), which is an external device. A schematic configuration of the endoscope 1 is as described above. Other components are assumed to be substantially the same as components of a conventional endoscope, and illustration and description are omitted.

Next, a detailed configuration of the bending operation mechanism 30 will be described below.

As shown in FIG. 3 and the like, the bending operation mechanism 30 includes the bending operation unit 31, a support member 37, and a restriction member 38 (see FIG. 5).

The bending operation unit 31 includes the bending operation lever 13, a rotator 35, the wire fixing member 36, and a coupling shaft 50.

The bending operation lever 13 includes a lever shaft 40 and a finger abutting member 41.

The lever shaft 40 extends from inside the operation section 3 to outside the operation section 3 through an opening 26 provided in a proximal end surface of the outer shell 10 of the operation section 3. The opening 26 is formed by combining a first opening portion 26a provided in the first outer shell 10a and a second opening portion 26b provided in the second outer shell 10b.

The finger abutting member 41 is connected to a proximal end of the lever shaft 40, and is disposed outside the operation section 3.

For example, the rotator 35 is a rotating member that is basically ball-shaped. A distal end of the lever shaft 40 of the bending operation lever 13 is connected to the rotator 35.

Furthermore, a recessed surface 45 is formed on a part of a surface of the rotator 35. The recessed surface 45 is shaped by cutting out a part of a spherical body of the rotator 35 in a V shape. The recessed surface 45 includes a first planar portion 45a and a second planar portion 45b. An angle formed by the first planar portion 45a and the second planar portion 45b is set to about 90 degrees (see FIG. 7), for example. The 90 degrees includes a design error, design flaw or range that may have the same effect or function as this disclosure. A valley line 45c that is formed by the first planar portion 45a and the second planar portion 45b intersecting each other is set at a position that passes through a center point P of the rotator 35 and that coincides with an extension line of a center axis Ax of the bending operation unit 31 (the lever shaft 40) (see FIGS. 3 and 4).

For example, as shown in FIGS. 3 to 6 and the like, the wire fixing member 36 is a substantially circular plate member. The substantially circular plate member includes a shape that may have the same effect or function as this disclosure. The coupling shaft 50 is provided protruding from a center of the wire fixing member 36. The coupling shaft 50 connects the wire fixing member 36 to the rotator 35 on an extension line of the center axis Ax of the lever shaft 40. Due to the configuration where the wire fixing member 36 and the rotator 35 are connected by the coupling shaft 50, the wire fixing member 36 is provided at a position away from the center point P of the rotator 35 (at a position different from the rotator 35) on the extension line extending in a direction (a longitudinal direction) along the center axis Ax of the lever shaft 40.

Furthermore, as shown in FIG. 6, a plurality of wire fixing parts (fixing parts) are provided near a periphery portion of one flat surface of the wire fixing member 36. In the example shown in FIG. 6, three wire fixing parts, that is, a first wire fixing part 51a, a second wire fixing part 51b, and a third wire fixing part 51c are provided as the plurality of wire fixing parts, for example. The plurality of wire fixing parts (51a, 51b, 51c) are formed as through holes that are provided at predetermined positions on a concentric circle at an equal distance from a center of the wire fixing member 36 in a radial direction, the predetermined positions being separated by mutually different predetermined distances in a circumferential direction. The proximal ends of the plurality of bending wires (25a, 25b, 25c) are fixed to the wire fixing parts (51a, 51b, 51c), respectively.

The support member 37 is a structural part that supports the bending operation unit 31 while allowing the bending operation unit 31 to swing within a predetermined range. For example, as shown in FIGS. 3 to 5 and the like, the support member 37 includes a first support portion 55 provided on an inner wall surface of the first outer shell 10a, and a second support portion 56 provided on an inner wall surface of the second outer shell 10b. The present embodiment describes an example where the first support portion 55 is integrally formed with the first outer shell 10a. In the same manner, an example is described where the second support portion 56 is integrally formed with the second outer shell 10b.

As shown in FIG. 4 and the like, the first support portion 55 is a protrusion that has a substantially circular columnar shape and that protrudes from the inner wall surface of the first outer shell 10a toward inside of the first outer shell 10a. A first spherical surface portion 57 having a recessed, partially spherical shape is formed on the first support portion 55. An inner diameter of the first spherical surface portion 57 is set to be slightly larger than an outer diameter of the rotator 35. Furthermore, the first support portion 55 includes a first communication groove 57a and a second communication groove 57b for allowing inside and outside of the first spherical surface portion 57 to communicate with each other. The first communication groove 57a and the second communication groove 57b are arc-shaped grooves having an inner diameter that is larger than a shaft diameter of each of the lever shaft 40 and the coupling shaft 50, for example. The first communication groove 57a and the second communication groove 57b are formed at positions facing each other across a center point of the first spherical surface portion 57.

As shown in FIG. 5 and the like, the second support portion 56 is a protrusion that has a substantially circular columnar shape and that protrudes from the inner wall surface of the second outer shell 10b toward inside of the second outer shell 10b. As in the case of the first support portion 55, a second spherical surface portion 58 having a recessed, partially spherical shape is formed on the second support portion 56. An inner diameter of the second spherical surface portion 58 is set to be equal to the inner diameter of the first spherical surface portion 57.

Accordingly, when the second support portion 56 is provided at a position at which the second support portion 56 exactly faces the first support portion 55 when the first outer shell 10a and the second outer shell 10b are joined together. Furthermore, the center point of the first spherical surface portion 57 and a center point of the second spherical surface portion 58 are set at positions that substantially coincide with each other when the first outer shell 10a and the second outer shell 10b are joined together.

Furthermore, the second support portion 56 includes a third communication groove 58a and a fourth communication groove 58b for allowing inside and outside of the second spherical surface portion 58 to communicate with each other. The third communication groove 58a and the fourth communication groove 58b are arc-shaped grooves having an inner diameter that is larger than the shaft diameter of each of the lever shaft 40 and the coupling shaft 50, for example. The third communication groove 58a and the fourth communication groove 58b are provided at positions that face each other across the center point of the second spherical surface portion 58, and that exactly face the first communication groove 57a and the second communication groove 57b.

With the support member 37 (the first support portion 55 and the second support portion 56) configured as described above, the rotator 35 is sandwiched between the first spherical surface portion 57 and the second spherical surface portion 58 when the first outer shell 10a and the second outer shell 10b are joined together. At least a part of the rotator 35 comes into internal contact with the first spherical surface portion 57 and the second spherical surface portion 58 through surface contact in a slidable manner. As described above, because the rotator 35 is in internal contact with the first spherical surface portion 57 and the second spherical surface portion 58 in a slidable manner, the rotator 35 is supported inside the operation section 3 while being rotatable within a predetermined range.

In the case described above, the lever shaft 40 penetrates through the first communication groove 57a and the third communication groove 58a. Furthermore, the coupling shaft 50 penetrates through the second communication groove 57b and the fourth communication groove 58b. As described above, the inner diameters of the first communication groove 57a and the third communication groove 58a are set to be larger than the shaft diameter of the lever shaft 40. The inner diameters of the second communication groove 57b and the fourth communication groove 58b are also set to be larger than the shaft diameter of the coupling shaft 50. Accordingly, the bending operation lever 13 and the wire fixing member 36 are freely tiltable relative to the support member 37 (the first support portion 55 and the second support portion 56).

The restriction member 38 is a structural member that is provided to restrict a rotation motion of the bending operation lever 13 (the lever shaft 40) around the center axis Ax, and to allow a tilting motion, within a predetermined range, of the bending operation lever 13 (the lever shaft 40) in every direction.

The restriction member 38 is formed by a flat plate member, a plan-view shape of which is a substantially right triangle. The restriction member 38 is integrally formed inside the second spherical surface portion 58. In the present case, a distal end of the substantially right triangle shape of the restriction member 38 is formed to protrude toward the center point of the second spherical surface portion 58 (see FIGS. 5 and 7).

As described above, the outer shell 10 as the housing of the operation section 3 is formed by combining the first outer shell 10a and the second outer shell 10b. In such a case, the rotator 35 is assembled by being sandwiched between the first spherical surface portion 57 of the first support portion 55 and the second spherical surface portion 58 of the second support portion 56. Here, as shown in FIG. 7, the restriction member 38 is inserted in the recessed surface 45 of the rotator 35, and is disposed with the distal end of the substantially right triangle protruding toward the center point P.

Accordingly, the restriction member 38 restricts the rotation motion of the bending operation lever 13 (the lever shaft 40) around the center axis Ax, and allows the tilting motion, within a predetermined range, of the bending operation lever 13 (the lever shaft 40) in every direction.

Accordingly, in the case where the bending operation lever 13 is tilted, only relative positions and angles between the restriction member 38 and the first planar portion 45a and the second planar portion 45b of the recessed surface 45 are changed, while a relative positional relationship between the center point P of the rotator 35 and the restriction member 38 remains substantially unchanged. The tilting motion of the bending operation lever 13 in every direction including forward-backward direction and left-right direction is thus allowed.

In the case where an amount of force in a rotation direction around the center axis Ax of the lever shaft 40 is applied to the bending operation lever 13, one of the first planar portion 45a or the second planar portion 45b abuts against the restriction member 38. The rotation motion of the bending operation lever 13 (the lever shaft 40) around the center axis Ax is thereby restricted.

As described above, when the bending operation lever 13 (the lever shaft 40) is built in at a predetermined position inside the operation section 3, the tilting motion in every direction is allowed within a predetermined range. When a tilting operation is performed on the bending operation lever 13, the rotator 35 rotates inside the first spherical surface portion 57 and the second spherical surface portion 58. At the time, because the restriction member 38 is inserted in the recessed surface 45 of the rotator 35, the restriction member 38 comes into contact with the recessed surface 45.

Accordingly, to secure smooth contact with the recessed surface 45, the restriction member 38 is formed such that a shape of a side surface portion of the flat plate member is curved in a thickness direction (see FIG. 5 and the like). Note that other shapes may also be adopted as long as smooth contact can be secured between the restriction member 38 and the recessed surface 45.

Note that, in the present embodiment, the restriction member 38 is integrally formed with the second spherical surface portion 58 of the second outer shell 10b, but such a mode is not restrictive. For example, the restriction member 38 may be integrally formed with the first spherical surface portion 57 of the first outer shell 10a. Furthermore, the restriction member 38 may be attached, as another member, to one of the first outer shell 10a or the second outer shell 10b, for example.

Moreover, the distal end of the restriction member 38 does not have to strictly face the center point of the second spherical surface portion 58. The distal end of the restriction member 38 may face a center direction of the rotator 35 with a certain margin.

Note that the restriction member 38 is such that, in a state where the bending operation lever 13 is at a neutral position shown in FIG. 8, a small gap (see reference sign W1) is present between the distal end of the restriction member 38 and the recessed surface 45, as shown in FIG. 9. For example, when the bending operation lever 13 is tilted in the manner shown in FIG. 10, the distal end of the restriction member 38 and the recessed surface 45 approach each other, and the gap W1 is eliminated (see reference sign W2), as shown in FIG. 11. Note that the examples shown in FIGS. 10 and 11 show a state where the bending operation lever 13 is tilted in a downward direction. However, the same can be said in the case of tilting the bending operation lever 13 in another direction.

Note that the state where the bending operation lever 13 is at the neutral position is a state where the bending operation lever 13 is not tilted. The bending operation lever 13 is kept at a predetermined position in a state where an amount of force from a hand or a finger, for example, is not applied by a user. The position of the bending operation lever 13 in such a state is referred to as the neutral position. When the bending operation lever 13 is at the neutral position, the bending portion 7 is kept in a substantially straight state with no bending motion in the up, down, left and right directions.

A setting as described above where a small gap is secured between the distal end of the restriction member 38 and the recessed surface 45 when the bending operation lever 13 is at the neutral position is for reliably achieving a tilting motion of the bending operation lever 13.

As described above, the proximal ends of the three bending wires (25a, 25b, 25c) are fixed, respectively, to the wire fixing parts (51a, 51b, 51c) of the wire fixing member 36 of the bending operation unit 31 (see FIGS. 3, 6, and the like).

As shown in FIG. 3 and the like, after extending from the respective wire fixing parts (51a, 51b, 51c), the three bending wires (25a, 25b, 25c) are guided by corresponding wire guide pins (wire guide) (32a, 32b, 32c), and are finally led to the insertion section 2. In such a case, the plurality of wire guide pins (32a, 32b, 32c) each function as a wire guide member that guides the corresponding bending wire (25a, 25b, 25c) while maintaining a state where tension is applied, and that changes an extension direction (also referred to as a pull direction) of the corresponding wire.

Here, three wire guide pins are provided, one each for the three bending wires. In other words, as the plurality of wire guide pins, there are a first wire guide pin 32a as a first wire guide, a second wire guide pin 32b as a second wire guide, and a third wire guide pin 32c as a third wire guide.

The plurality of wire guide pins (32a, 32b, 32c) are each formed as a member that has a substantially circular columnar shape or a substantially cylindrical shape, and that protrudes inward from the inner wall surface of the first outer shell 10a, for example. The plurality of wire guide pins (32a, 32b, 32c) are integrally formed with the outer shell 10, for example.

For example, the plurality of wire guide pins are assumed to be integrally formed with the outer shell 10 by integral molding. More specifically, for example, a mode is conceivable where the plurality of wire guide pins are formed as a part of the outer shell 10 by molding. Such a configuration may contribute to reduction in a manufacturing cost.

Furthermore, pin-shaped members that are parts separate from the outer shell 10 may be attached and fixed, as the plurality of wire guide pins, to the inner wall surface of the outer shell 10 by fixing means such as screwing.

A plurality of boss members (fourth wire guide) 33 are provided near the plurality of wire guide pins (32a, 32b, 32c). The plurality of boss members 33 are boss members for screwing used at the time of joining the first outer shell 10a and the second outer shell 10b and forming the housing of the operation section 3. The plurality of boss members 33 are formed by members having substantially the same form as the plurality of wire guide pins. Note that the present embodiment shows an example where two boss members 33 are provided.

Furthermore, like the plurality of wire guide pins (32a, 32b, 32c) described above, a part of the boss members 33 is used also as a wire guide member that guides each bending wire (25a, 25b, 25c) while maintaining a state where tension is applied, changes an extension direction of each wire, and finally leads the wire to the insertion section 2.

As described above, as shown in FIGS. 3, 4 and the like, each of the bending wires (25a, 25b, 25c) that are led to the insertion section 2 is guided from the wire fixing member 36 in a state where tension is applied by the plurality of wire guide pins (32a, 32b, 32c) and a part of the boss members 33.

Note that after being guided by a part of the boss members 33 and having an extension direction changed, the plurality of bending wires (25a, 25b, 25c) are inserted through a plurality of guide members (34a, 34b, 34c) held by the first outer shell 10a to extend to inside of the insertion section 2. Each bending wire (25a, 25b, 25c) is thus inserted up to a distal end of the bending portion 7 through the insertion section 2. Lastly, a distal end of each bending wire (25a, 25b, 25c) is fixed at a predetermined position on a distal end side of the bending tube 20 of the bending portion 7.

With the endoscope 1 of the present embodiment, the wire fixing parts (51a, 51b, 51c) of the wire fixing member 36 and the plurality of wire guide pins (32a, 32b, 32c) are each arranged in the following manner.

First, as shown in FIG. 13, in a state where the bending operation unit 31 at the neutral position is seen in an arrow [13] direction in FIG. 12, a horizontal axis orthogonal to the center axis Ax of the bending operation unit 31 is given as an X-axis. A consideration is given on an XY-plane that is obtained by defining a Y-axis orthogonal to the X-axis. An intersection point of the X-axis, the Y-axis, and the center axis Ax of the bending operation unit 31 is given as a point of origin (0, 0) of the XY-plane. The operation section 3 for use with the endoscope 1 can comprise the shaft (40, 50), the wire fixing surface 36 connected to the shaft (40, 50), the first wire 25a, the second wire 25b, and the third wire 25c. Each of the first through third wires has the fixing part (51a, 51b, 51c) attached to the wire fixing surface 36. The first wire guide 32a is configured to guide the first wire 25a. The second wire guide 32b is configured to guide the second wire 25b. The third wire guide 32c is configured to guide the third wire 25c. The first wire 25a includes a first portion between the fixing part 51a of the first wire 25a and the first wire guide 32a. The second wire 25b includes a second portion between the fixing part 51b of the second wire 25b and the second wire guide 32b. In a view from a neutral axis of the shaft (40, 50), the first and second portions cross. The third wire 25c can include a third portion between the fixing part 51c of the third wire 25c and the third wire guide 32c, in the view from the neutral axis of the shaft (40, 50), the third portion crosses the first portion and second portion. In the view from the neutral axis of the shaft (40, 50), the fixing part 51a of the first wire 25a can be located between the second portion of the second wire and the third portion of the third wire 25c. In the view from the neutral axis of the shaft (40, 50), the first portion of the first wire 25a can be located between the fixing part 51a of the second wire 25b and the fixing part 51c of the third wire 25c. The neutral axis can be an axis of the shaft (i) when the shaft is positioned at the neutral position, (ii) when the shaft is not operated by the user, or (iii) when the shaft is not applied an operation force.

In other words, in the present embodiment, the X-axis is an axis in a tilting direction in which the bending operation lever 13 is tilted to bend the bending portion 7 in the left-right direction. Furthermore, the Y-axis is an axis in a tilting direction in which the bending operation lever 13 is tilted to bend the bending portion 7 in the up-down direction.

On the XY-plane, four parts divided by the X-axis and the Y-axis will be referred to as a first quadrant [I], a second quadrant [II], a third quadrant [III], and a fourth quadrant [IV] from top right, top left, bottom left, and bottom right.

The proximal ends of the plurality of bending wires (25a, 25b, 25c) are fixed to the wire fixing member 36 while being disposed in different quadrants from one another on the XY-plane.

More specifically, for example, as shown in FIG. 13, the first wire fixing part 51a where the proximal end of the first bending wire 25a is fixed is disposed in the fourth quadrant [IV]. Furthermore, as shown in FIG. 13, the first wire guide pin 32a for changing a wire extension direction of the first bending wire 25a is disposed in the first quadrant [I].

In other words, in the present case, the first wire guide pin 32a (the first wire guide) guides the first bending wire 25a (the first wire) at a position (the first quadrant) on an opposite side from the proximal end (the fourth quadrant) of the first bending wire 25a (the first wire) across the X-axis on the XY-plane.

Furthermore, for example, as shown in FIG. 13, the second wire fixing part 51b where the proximal end of the second bending wire 25b is fixed is disposed in the second quadrant [II]. Furthermore, as shown in FIG. 13, the second wire guide pin 32b for changing a wire extension direction of the second bending wire 25b is disposed in the fourth quadrant [IV].

In other words, in the present case, the second wire guide pin 32b (the second wire guide) guides the second bending wire 25b (the second wire) at a position (the fourth quadrant) on an opposite side from the proximal end (the second quadrant) of the second bending wire 25b (the second wire) across both the X-axis and the Y-axis on the XY-plane.

Furthermore, for example, as shown in FIG. 13, the third wire fixing part 51c where the proximal end of the third bending wire 25c is fixed is disposed in the first quadrant [I]. Furthermore, as shown in FIG. 13, the third wire guide pin 32c for changing a wire extension direction of the third bending wire 25c is disposed in the third quadrant [III].

In other words, in the present case, the third wire guide pin 32c (the third wire guide) guides the third bending wire 25c (the third wire) at a position (the third quadrant) on an opposite side from the proximal end (the first quadrant) of the third bending wire 25c (the third wire) across both the X-axis and the Y-axis on the XY-plane.

The plurality of wire guide pins (32a, 32b, 32c) are disposed at positions that are separated from a movement range of the wire fixing member 36 that moves in conjunction with tilting of the bending operation lever 13. In FIG. 12 and the like, a dash-dot-dotted line indicated by a reference sign M indicates the movement range of the wire fixing member 36 that moves in conjunction with tilting of the bending operation lever 13. The plurality of wire guide pins (32a, 32b, 32c) are each disposed at a position that does not interfere with the movement range M.

In other words, when a fulcrum (the center point P of the rotator 35) of tilting of the bending operation lever 13 under an operation force from a finger, a hand or the like of a user is given as a center, the movement range M is defined by a part of a sphere that is given by taking, as a radius, a distance between the fulcrum (reference sign P) and the proximal end (51a, 51b, 51c) of at least one of the plurality of bending wires (25a, 25b, 25c). Here, the plurality of wire guide pins (32a, 32b, 32c) are disposed at positions outside the sphere that is given (the dash-dot-dotted line M). The first wire 25a can include a first contact point contacting with the first wire guide 32a, the second wire 25b can include a second contact point contacting with the second wire guide 32b, and the third wire 25c can include a third contact point contacting with the third wire guide 32c. In the view from the neutral axis of the shaft (40, 50), the first to the third contact points don't overlap each other. In a view from a direction perpendicular to the neutral axis of the shaft (40, 50), the first to the third contact points don't overlap each other. The first to the third wire guides (32a, 32b, 32c) can extend along the direction perpendicular to the neutral axis of the shaft (40, 50). The operation section 3 can comprise the fourth wire guide 33 configured to guide the first to third wires (25a, 25b, 25c), the fourth wire guide 33 extending along the direction perpendicular to the neutral axis of the shaft (40, 50). The first to the fourth wire guides can be spaced apart from each other. The axes of the first to the fourth wire guides can be spaced apart from each other.

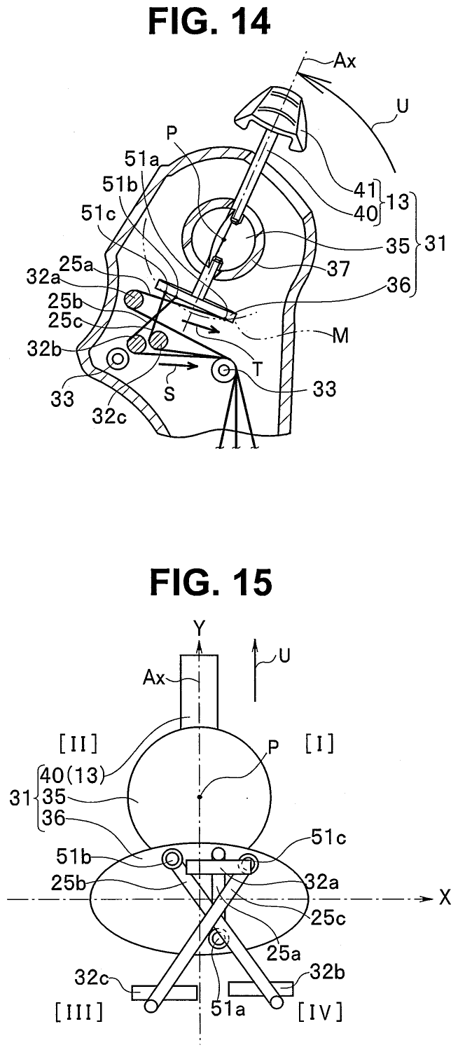

With respect to the endoscope 1 of the present embodiment configured in the above manner, operation of the bending operation unit 31 and the bending operation mechanism 30 will be described below with reference to FIGS. 14 to 19.

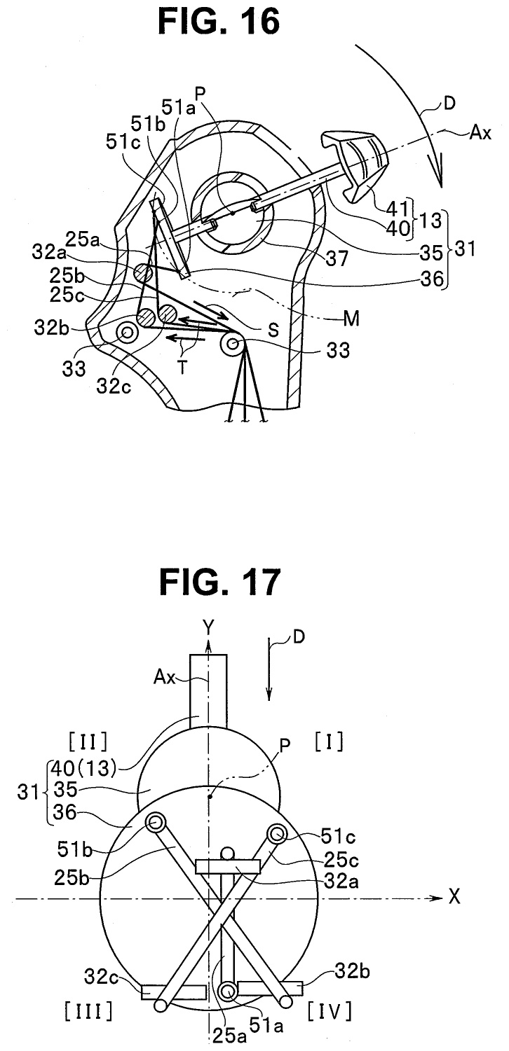

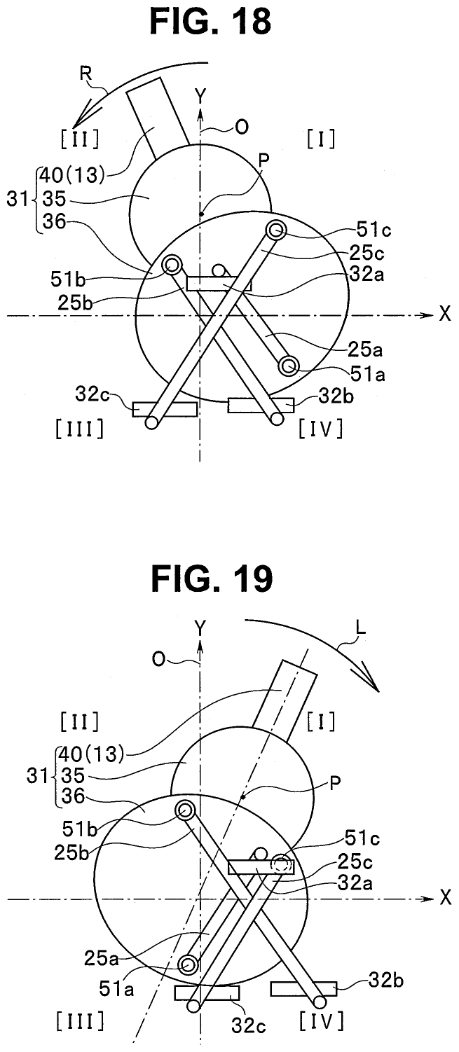

FIGS. 14 and 15 are diagrams showing a state of the bending operation unit and the plurality of bending wires when the bending operation lever is tilted in an upward direction (arrow U direction). FIGS. 16 and 17 are diagrams showing a state of the bending operation unit and the plurality of bending wires when the bending operation lever is tilted in a downward direction (arrow D direction). FIG. 18 is a diagram showing a state of the bending operation unit and the plurality of bending wires when the bending operation lever is tilted in a right direction (arrow R direction). FIG. 19 is a diagram showing a state of the bending operation unit and the plurality of bending wires when the bending operation lever is tilted in a left direction (arrow L direction).

Note that, like FIG. 12, FIGS. 14 and 16 are vertical cross-sectional views showing a state of the bending operation unit and the plurality of bending wires when the operation section is seen from a side (only the outer shell is shown in cross-section). Furthermore, like FIG. 13, FIGS. 15, 17, 18, and 19 conceptually show a state of the bending operation unit and the plurality of bending wires seen from a front of the operation section.

First, the bending operation unit 31 (13, 35, 36, 50, etc.) of the endoscope 1 is assumed to be at the neutral position shown in FIGS. 12 and 13. In the state where the bending operation unit 31 is at the neutral position, a user of the endoscope 1 applies a predetermined amount of force on the bending operation lever 13 of the bending operation unit 31 in a predetermined direction corresponding to a predetermined bending direction. Then, the bending operation lever 13 is tilted by a predetermined amount in the predetermined direction. Accordingly, a predetermined wire among the plurality of bending wires (25a, 25b, 25c) is pulled by a predetermined amount as appropriate. As a result, the bending portion 7 is bent by a predetermined amount in the predetermined direction by the user.

For example, in the case where bending of the bending portion 7 in the upward direction (UP direction) is performed, the bending operation lever 13 is tilted in the arrow U direction, as shown in FIGS. 14 and 15. Then, the wire fixing member 36 is moved in a predetermined direction in conjunction with the tilting motion. Here, the bending operation lever 13 rotates in a counterclockwise direction around the center point P of the rotator 35 in FIG. 14.

Accordingly, the first bending wire 25a, the proximal end of which is fixed to the first wire fixing part 51a, is pulled in an arrow T direction. At the same time, the second bending wire 25b, the proximal end of which is fixed to the second wire fixing part 51b, and the third bending wire 25c, the proximal end of which is fixed to the third wire fixing part 51c, both move in an arrow S direction in FIG. 14, and each wire 25b, 25c is loosened and is not pulled. Accordingly, here, only the first bending wire 25a is pulled. The bending portion 7 can thus be bent in the upward direction (the UP direction). When the shaft (40, 50) is tilted relative to the neutral axis, one of the first to the third wire (32a, 32b, 32c) may not be tensioned.

For example, in the case where bending of the bending portion 7 in the downward direction (DOWN direction) is performed, the bending operation lever 13 is tilted in the arrow D direction, as shown in FIGS. 16 and 17. Then, the wire fixing member 36 is moved in a predetermined direction in conjunction with the tilting motion. Here, the bending operation lever 13 rotates in a clockwise direction around the center point P of the rotator 35 in FIG. 16.

Accordingly, the first bending wire 25a moves in the arrow S direction in FIG. 16, and the first bending wire 25a is loosened and is not pulled. At the same time, the second bending wire 25b and the third bending wire 25c are both pulled in the arrow T direction in FIG. 16. Accordingly, here, the second bending wire 25b and the third bending wire 25c are pulled. The bending portion 7 can thus be bent in the downward direction (the DOWN direction).

For example, in the case where bending of the bending portion 7 in the right direction (RIGHT direction) is performed, the bending operation lever 13 is tilted in the arrow R direction, as shown in FIG. 18. Then, the wire fixing member 36 is moved in a predetermined direction in conjunction with the tilting motion. Here, the bending operation lever 13 rotates in a counterclockwise direction around the center point P of the rotator 35 in FIG. 18.

The first bending wire 25a and the third bending wire 25c are thereby pulled. At the same time, the second bending wire 25b is loosened and is not pulled. Accordingly, only the first bending wire 25a and the third bending wire 25c are pulled. The bending portion 7 can thus be bent in the right direction (the RIGHT direction).

Furthermore, for example, in the case where bending of the bending portion 7 in the left direction (LEFT direction) is performed, the bending operation lever 13 is tilted in the arrow L direction, as shown in FIG. 19. Then, the wire fixing member 36 is moved in a predetermined direction in conjunction with the tilting motion. Here, the bending operation lever 13 rotates in a clockwise direction around the center point P of the rotator 35 in FIG. 19.

The first bending wire 25a and the second bending wire 25b are thereby pulled. At the same time, the third bending wire 25c is loosened and is not pulled. Accordingly, only the first bending wire 25a and the second bending wire 25b are pulled. The bending portion 7 can thus be bent in the left direction (the LEFT direction).

Note that a range (operation movable range) in which the bending operation lever 13 can be tilted when the bending operation lever 13 is tilted in any direction including the up, down, left and right directions is restricted by the opening 26 in the proximal end surface of the outer shell 10, or by the first communication groove 57a and the second communication groove 57b of the first spherical surface portion 57 and the third communication groove 58a and the fourth communication groove 58b of the second spherical surface portion 58. In other words, a tilting range of the bending operation lever 13 is restricted to a predetermined region.

Accordingly, with the bending operation mechanism 30 of the present embodiment, in a movable range of the bending operation lever 13, a loosened or pulled state of each bending wire (25a, 25b, 25c) is not changed and is maintained even when the bending operation lever 13 is greatly tilted.

As described above, according to the embodiment described above, the insertion device, such as the endoscope 1, including the bending operation mechanism 30 for achieving a predetermined bending motion of the bending portion 7 by pulling one or two bending wires among the plurality of bending wires (25a, 25b, 25c) in conjunction with a tilting operation on the joystick-type bending operation lever 13 includes a plurality of independent wire guide pins (32a, 32b, 32c), one each for the plurality of bending wires (25a, 25b, 25c).

According to such a configuration, an amount of pull on each bending wire (25a, 25b, 25c) to achieve a predetermined bending direction and a predetermined bending amount in relation to the bending portion 7 can be optimized. Accordingly, a stable and highly accurate bending motion can constantly be achieved.

### [Modifications]

With the endoscope 1 according to the embodiment described above, the plurality of wire guide pins (32a, 32b, 32c) are formed by attaching substantially circular columnar or cylindrical members to the outer shell 10 by screwing or by integrally molding such members with the outer shell 10.

Various modifications as described below are further conceivable in relation to the plurality of wire guide pins (32a, 32b, 32c).

### [First Modification]

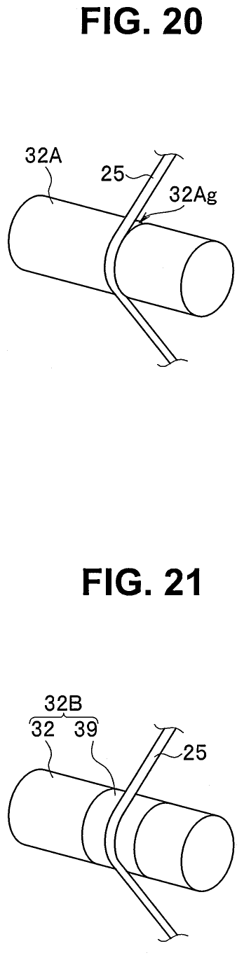

FIG. 20 is a diagram showing a first modification of the endoscope of the embodiment of the present disclosure. More specifically, the first modification is a modification related to the wire guide pin of the bending operation mechanism included in the endoscope of the embodiment of the present disclosure.

As shown in FIG. 20, a wire guide pin 32A of the first modification includes a peripheral groove 32Ag. The peripheral groove 32Ag is a wire reception portion configured to receive the bending wire (see reference sign 25 in FIG. 20), and is a wire guide groove.

With the wire guide pin 32A of the first modification, the bending wire 25 is received in the peripheral groove 32Ag, and thus, when a pull force is applied to the bending wire 25 and the bending wire 25 slides on a peripheral surface of the wire guide pin 32A, the bending wire 25 is prevented from moving in an axial direction of the wire guide pin 32A. The bending wire 25 can thereby be reliably guided.

### [Second Modification]

FIG. 21 is a diagram showing a second modification of the endoscope of the embodiment of the present disclosure. More specifically, the second modification is a modification related to the wire guide pin of the bending operation mechanism included in the endoscope of the embodiment of the present disclosure.

As shown in FIG. 21, a wire guide pin 32B of the second modification includes a pin member (see reference sign 32 in FIG. 21) and a ring 39. Like the wire guide pin in the embodiment described above, the pin member 32 is a substantially circular columnar or cylindrical member.

The ring 39 is a wire reception portion that is formed by using a low friction member formed from fluororesin or the like, and that has a substantially annular shape. The ring 39 is integrated with the pin member 32 by being lightly pressed and joined, for example.

With the wire guide pin 32B of the second modification, because the ring 39 that is a low friction member is disposed on a peripheral surface of the pin member 32, smooth sliding of the bending wire 25 on the wire guide pin 32B can be secured, and an amount of bending operation force can be reduced. Moreover, because the ring 39 is assembled by being lightly pressed and joined, for example, an assembly cost can be reduced.

### [Third Modification]

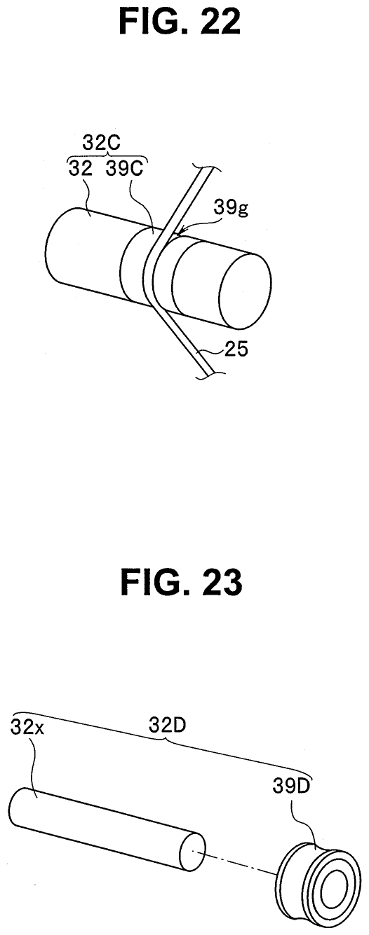

Note that, in contrast to the ring 39 of the second modification, it is also possible to form a wire guide pin 32C including a ring 39C including a wire guide groove 39g that is similar to the peripheral groove of the first modification, as shown in FIG. 22.

Such a configuration can achieve a same effect, and can also achieve a same effect as the effect of the first modification.

FIG. 22 is a diagram showing a third modification of the endoscope of the embodiment of the present disclosure. More specifically, the third modification is a modification related to the wire guide pin of the bending operation mechanism included in the endoscope of the embodiment of the present disclosure.

As shown in FIG. 22, the wire guide pin 32C of the third modification adopts the ring 39C that includes the wire guide groove 39g.

With the wire guide pin 32C of the third modification, the bending wire 25 can be more smoothly and stably guided.

### [Fourth Modification]

FIG. 23 is a diagram showing a fourth modification of the endoscope of the embodiment of the present disclosure. More specifically, the fourth modification is a modification related to the wire guide pin of the bending operation mechanism included in the endoscope of the embodiment of the present disclosure.

As shown in FIG. 23, a wire guide pin 32D of the fourth modification includes a pin member 32x and a bearing member 39D. Substantially like the wire guide pin of the embodiment described above, the pin member 32x is a substantially circular columnar or cylindrical member.

Furthermore, the bearing member 39D is a wire reception portion such as a roller bearing, a pulley component, or a pulley, for example. The bearing member 39D is integrated with the pin member 32x by being lightly pressed and joined, for example.

With the wire guide pin 32D of the fourth modification configured in the above manner, the bending wire 25 can be stably guided as in the case of the first modification described above, and also, the amount of operation force and the assembly cost can be further reduced than in the second modification and the third modification described above.

### [Fifth Modification]

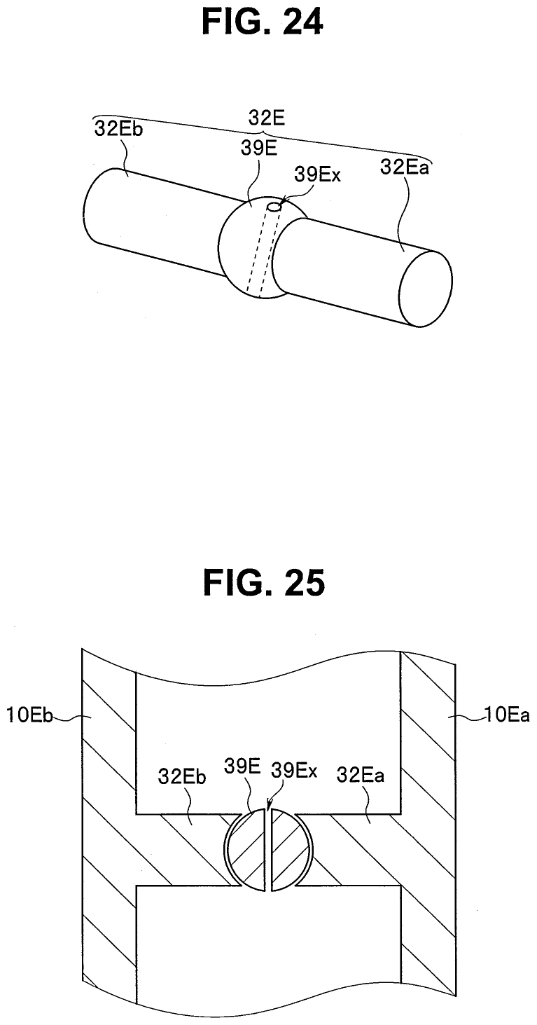

FIGS. 24, 25, and 26 are diagrams showing a fifth modification of the endoscope of the embodiment of the present disclosure. More specifically, the fifth modification is a modification related to the wire guide pin of the bending operation mechanism included in the endoscope of the embodiment of the present disclosure.

As shown in FIG. 24 and the like, a wire guide pin 32E of the fifth modification includes a ball 39E, a first boss member 32Ea, and a second boss member 32Eb.

The ball 39E is a spherical member including a through hole 39Ex that allows insertion of the bending wire 25. The first boss member 32Ea and the second boss member 32Eb are support members configured to rotatably support the ball 39E.

The first boss member 32Ea is a substantially circular columnar or cylindrical member that protrudes inward from the inner wall surface of the first outer shell 10a. A semi-spherical ball reception portion configured to receive the ball 39E is formed on a distal end of the first boss member 32Ea.

Likewise, the second boss member 32Eb is a substantially circular columnar or cylindrical member that protrudes inward from the inner wall surface of the second outer shell 10b. A semi-spherical ball reception portion configured to receive the ball 39E is formed on a distal end of the second boss member 32Eb.

Furthermore, as shown in FIG. 26, the first boss member 32Ea and the second boss member 32Eb are disposed at positions that face each other when the first outer shell 10a and the second outer shell 10b are joined together. Accordingly, due to such a configuration, when the first outer shell 10a and the second outer shell 10b are joined together, the ball 39E is rotatably supported between (the distal end reception portion of) the first boss member 32Ea and (the distal end reception portion of) the second boss member 32Eb, as shown in FIG. 25. Each bending wire (25a, 25b, 25c) is inserted through the through hole 39Ex in the ball 39E.

Note that the first boss member 32Ea and the second boss member 32Eb are integrally formed with the first outer shell 10a and the second outer shell 10b, respectively. In such a case, the first boss member 32Ea and the second boss member 32Eb may each be formed by being integrally molded with the corresponding outer shell (10a, 10b), or a member that is separately formed may be joined to the outer shell by being joined with screws or the like.

Furthermore, a position of each ball 39E in an axial direction of the wire guide pin 32E may be appropriately set by changing length of each of the first boss member 32Ea and the second boss member 32Eb.

As shown in FIG. 26, with the example configuration described in the fifth modification, a wire guide groove 33Eg is provided on an outer peripheral surface of a boss member 33E that serves also as a wire guide member. The bending wire can thereby be more stably guided.

With the wire guide pin 32E of the fifth modification configured in the above manner, when the bending wire (25a, 25b, 25c) is pulled in conjunction with the tilting operation on the bending operation unit 31, the ball 39E rotates in conjunction with the bending wire, and friction between the bending wire and the wire guide pin 32E may be reduced.

Furthermore, when a pull force is applied to the bending wire (25a, 25b, 25c), movement of the bending wire 25 in the axial direction of the wire guide pin 32E may be prevented. Accordingly, the bending wire (25a, 25b, 25c) can be reliably guided.

Note that the configuration of the fifth modification does not have to be adopted by all of the plurality of wire guide pins as long as the configuration is adopted by at least one of the plurality of wire guide pins.

The bending operation mechanism applied to the endoscope of the embodiment described above includes the joystick-type bending operation lever, and any one of the three bending wires is pulled in conjunction with the tilting operation on the bending operation lever to thereby achieve a bending motion of the bending portion in the up, down, left and right directions.

By contrast, a conventional bending operation mechanism generally adopts a mechanism including four bending wires, one each for four directions of the up, down, left and right directions of the bending portion, for example. With the conventional bending operation mechanism including four bending wires, an amount of pull on the wire can be comparatively easily adjusted by separately adjusting each bending wire provided for each bending direction, to achieve a bending operation in a predetermined bending direction and with a predetermined bending amount.

In contrast to such a conventional bending operation mechanism including four bending wires, with a configuration that uses three bending wires to achieve a bending motion of the bending portion in the up, down, left and right directions as in the case of the bending operation mechanism of the endoscope of the embodiment described above, a balance among the amounts of pull on the three bending wires has to be appropriately adjusted at all times to accurately and reliably achieve a bending operation in a predetermined bending direction and with a predetermined bending amount.

Accordingly, in relation to the bending operation mechanism that may achieve a bending motion of the bending portion in the up, down, left and right directions by using three bending wires, a bending operation mechanism including a configuration that allows a balance among the amounts of pull on the three bending wires to be appropriately adjusted at all times will be described below.

### [Sixth Modification]

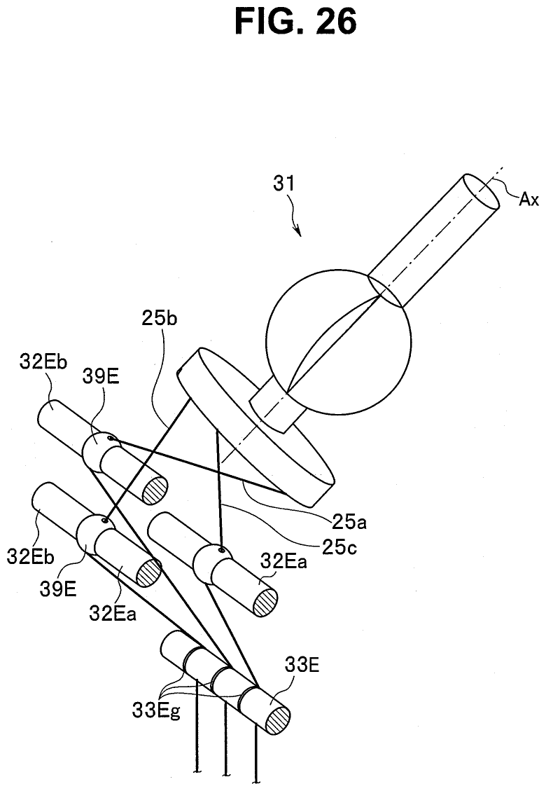

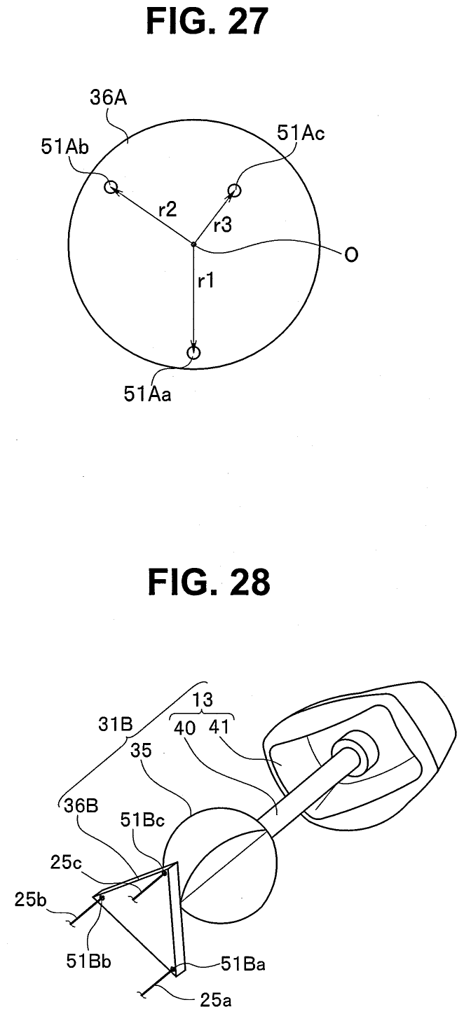

FIG. 27 is a diagram showing a sixth modification of the endoscope of the embodiment of the present disclosure. More specifically, the sixth modification is a modification related to arrangement of the wire fixing parts of the wire fixing member of the bending operation mechanism included in the endoscope of the embodiment of the present disclosure.

FIG. 27 shows a surface of a wire fixing member 36A where a plurality of wire fixing parts, to which a plurality of bending wires are fixed, are provided.

As described above, the plurality of wire fixing parts (51a, 51b, 51c) of the wire fixing member 36 of the bending operation mechanism 30 included in the endoscope 1 of the embodiment described above are provided at predetermined positions on a concentric circle at an equal distance from a center point O of the wire fixing member 36 in a radial direction, the predetermined positions being separated by mutually different predetermined distances in a circumferential direction.

In contrast to the configuration of the embodiment described above, in the sixth modification, a plurality of wire fixing parts (51Aa, 51Ab, 51Ac) are provided on different circles in a radial direction from a center point O of the wire fixing member 36A, as shown in FIG. 27. Also in the sixth modification, the plurality of wire fixing parts (51Aa, 51Ab, 51Ac) are provided at predetermined positions that are separated by mutually different predetermined distances in a circumferential direction. In the example shown in FIG. 27, a first wire fixing part 51Aa is provided on a circle with a radius r1. A second wire fixing part 51Ab is provided on a circle with a radius r2. A third wire fixing part 51Ac is provided on a circle with a radius r3.

Here, with respect to the positions of the plurality of wire fixing parts (51Aa, 51Ab, 51Ac), numerical values of radii r1, r2, r3 may be set as appropriate according to the mode of the bending operation mechanism, for example.

By setting mutually different distances as the distances between the center point O of the wire fixing member 36A and the positions of the plurality of wire fixing parts (51Aa, 51Ab, 51Ac), the amounts of pull on the three bending wires may be appropriately adjusted in a well-balanced manner.

Now, the embodiment described above describes an example where the wire fixing member 36 is a plate member, a planar shape of which is a substantially circular shape, and the plurality of wire fixing parts (51a, 51b, 51c) are formed as through holes. However, the mode of the wire fixing member is not limited to the example of the embodiment described above. Various modifications as described below are conceivable in relation to the wire fixing member.

### [Seventh Modification]

FIG. 28 is a diagram showing a seventh modification of the endoscope of the embodiment of the present disclosure. More specifically, the seventh modification is a modification related to the wire fixing member of the bending operation mechanism included in the endoscope of the embodiment of the present disclosure.

The seventh modification shown in FIG. 28 is an example where a planar shape of a wire fixing member 36B of a bending operation unit 31B is a polygonal shape. The seventh modification is an example where the planar shape of the wire fixing member 36B is made, for example, as an equilateral triangle to match the three bending wires.

In the present case, a plurality of wire fixing parts (51Ba, 51Bb, 51Bc) are provided at (near) three vertices of the wire fixing member 36B, respectively. As described above, the planar shape of the wire fixing member 36B is an equilateral triangle, and thus, distances from a center of the wire fixing members 36B to the plurality of wire fixing parts (51Ba, 51Bb, 51Bc) provided at (near) the respective vertices of the wire fixing member 36B can be set to be the same.

With the wire fixing member 36B of the seventh modification configured in the above manner, a number of wire fixing parts may be provided in a smaller area than in the case of the wire fixing member that is formed into a substantially circular shape, and a material cost can be reduced.

### [Eighth Modification]

Note that, by further improving the planar shape of the wire fixing member 36B of the seventh modification described above, a configuration substantially the same as the mode shown in the sixth modification described above (the mode where the wire fixing positions in the radial direction are made different) can be achieved.

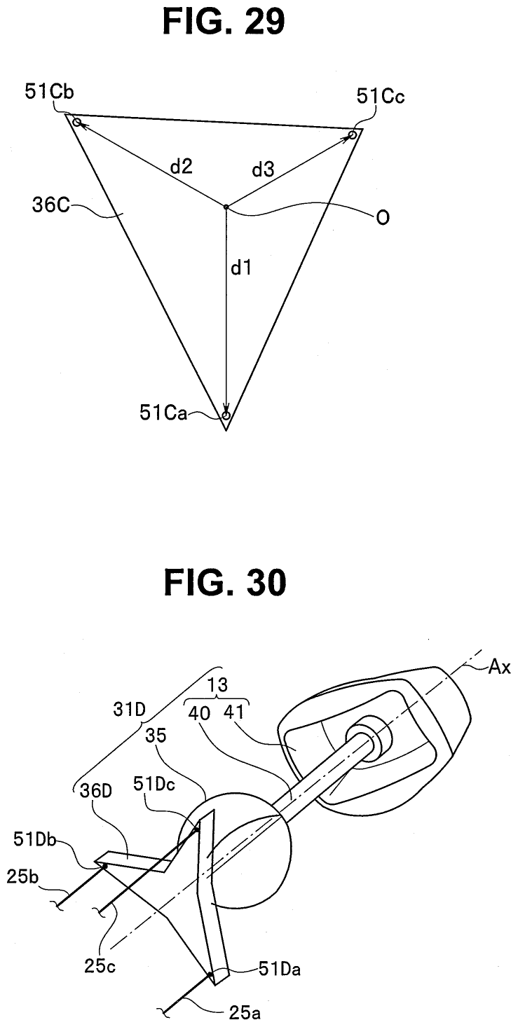

FIG. 29 is a diagram showing an eighth modification of the endoscope of the embodiment of the present disclosure. More specifically, the eighth modification is a modification related to the wire fixing member of the bending operation mechanism included in the endoscope of the embodiment of the present disclosure.

As shown in FIG. 29, a wire fixing member 36C of the eighth modification is formed into a triangular shape, as in the seventh modification described above. However, with the wire fixing member 36C of the eighth modification, distances from a center of gravity of the triangle (a center point O of the wire fixing member 36C) to the respective vertices are made different. In other words, a planar shape of the wire fixing member 36C of the eighth modification is an inequilateral triangle.

With the wire fixing member 36C having such a shape, distances from the center point O of the wire fixing member 36C to three wire fixing parts (51Ca, 51Cb, 51Cc) are set to be mutually different distances. In the example shown in FIG. 29, a first wire fixing part 51Ca is provided at a position that is separate from the center point O of the wire fixing member 36C by a distance d1. A second wire fixing part 51Cb is provided at a position that is separate from the center point O of the wire fixing member 36C by a distance d2. A third wire fixing part 51Cc is provided at a position that is separate from the center point O of the wire fixing member 36C by a distance d3. Here, d1 #d2 #d3 is true for the distances.

With the wire fixing member 36C of the eighth modification configured in the above manner, the same effect as the effect of the seventh modification described above may be obtained, and also, the same effect as the effect of the sixth modification described above may be obtained.

### [Ninth Modification]

FIG. 30 is a diagram showing a ninth modification of the endoscope of the embodiment of the present disclosure. More specifically, the ninth modification is a modification related to the wire fixing member of the bending operation mechanism included in the endoscope of the embodiment of the present disclosure.

As shown in FIG. 30, a wire fixing member 36D of the ninth modification includes the same number of arm portions as the plurality of bending wires (25a, 25b, 25c). A proximal end of each arm portion is provided at a center position of the wire fixing member 36D. A coupling shaft (not shown; a shaft that coincides with the center axis Ax) of a bending operation unit 31D is fixed at the center position of the wire fixing member 36D. The bending operation unit 31D is disposed upright relative to a plane of the wire fixing member 36D.

A distal end of each arm portion of the wire fixing member 36D extends in a radial direction. In the present case, the arm portions are formed at mutually different angles in a circumferential direction. The distal end of each arm portion is formed into a tapered shape that becomes narrower at the distal end. Moreover, a wire fixing part (51Da, 51Db, 51Dc) is formed near the distal end of each arm portion.

With the wire fixing member 36D of the ninth modification configured in the above manner, a number of wire fixing parts may be provided in an even smaller area. Accordingly, the material cost may be further reduced.

Furthermore, according to the configuration of the ninth modification, a distance from a center point of the wire fixing member 36D to each wire fixing part may be set to a predetermined length as appropriate simply by adjusting and forming a length of each arm portion as appropriate.

Furthermore, with the wire fixing member 36D, because extension directions and extension lengths of the arm portions from the center point may be freely set, a greater degree of freedom may be obtained in relation to arrangement of the wire fixing parts compared to the wire fixing members having polygonal shapes (triangular shapes) described in the seventh and eighth modifications described above.

### [Tenth Modification]

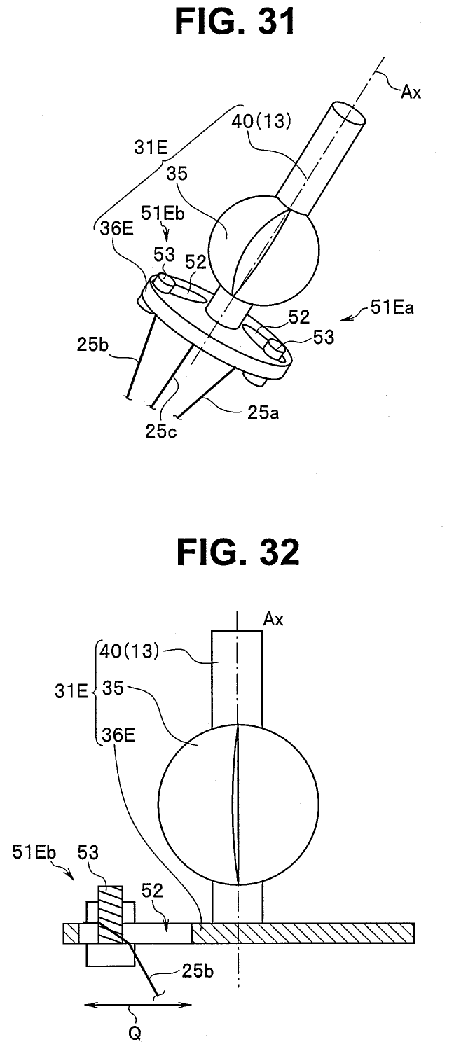

FIGS. 31 and 32 are diagrams showing a tenth modification of the endoscope of the embodiment of the present disclosure. More specifically, the tenth modification is a modification related to the wire fixing member of the bending operation mechanism included in the endoscope of the embodiment of the present disclosure.

Of the drawings, FIG. 31 is a perspective view of a bending operation unit of the tenth modification. FIG. 32 is a side cross-sectional view of the bending operation unit of the tenth modification. Note that FIG. 32 shows only the wire fixing member in cross-section.

As shown in FIGS. 31 and 32, a wire fixing member 36E of a bending operation unit 31E of the tenth modification is a substantially circular plate member as in the case of the embodiment described above. The wire fixing member 36E includes a plurality of wire fixing parts (see reference signs 51Ea and 51Eb in FIG. 31). In the present embodiment, the plurality of wire fixing parts are a first wire fixing part 51Ea, a second wire fixing part 51Eb, and a third wire fixing part (not shown).

Each wire fixing part (51Ea, 51Eb, etc.) includes a long hole 52 for position adjustment and bolt/nut 53 for fixing.

The long hole 52 for position adjustment is a long hole that extends in a radial direction from a center of the wire fixing member 36E and that penetrates the wire fixing member 36E, the long hole 52 for position adjustment having a predetermined width.

The bolt/nut 53 for fixing is a structural member that is inserted through the long hole 52 for position adjustment, and that fixes and holds corresponding one bending wire among the plurality of bending wires (25a, 25b, 25c). The bending wire is fixed and held by fastening the bolt/nut 53 for fixing in a state where the bending wire is sandwiched between the bolt/nut 53 for fixing and a surface of the wire fixing member 36E.

According to such a configuration, the bolt/nut 53 for fixing may be disposed in the long hole 52 for position adjustment, at any position within a range in the radial direction (direction along an arrow Q in FIG. 32) from a center axis Ax of the wire fixing member 36E.

Accordingly, with the tenth modification having such a configuration, a fixed position of the bending wire (25a, 25b, 25c) on the wire fixing member 36E may be freely set in the radial direction from the center axis Ax.

### [Eleventh Modification]

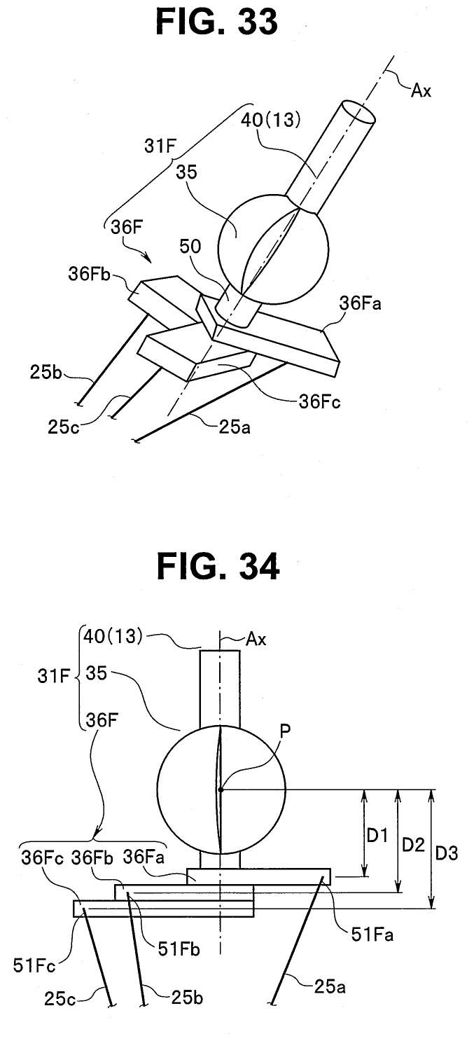

FIGS. 33 and 34 are diagrams showing an eleventh modification of the endoscope of the embodiment of the present disclosure. More specifically, the eleventh modification is a modification related to the wire fixing member of the bending operation mechanism included in the endoscope of the embodiment of the present disclosure.

Of the drawings, FIG. 33 is a perspective view of a bending operation unit of the eleventh modification. FIG. 34 is a side view of the bending operation unit of the eleventh modification.

As shown in FIGS. 33 and 34, a wire fixing member 36F of the eleventh modification includes a plurality of arm members (36Fa, 36Fb, 36Fc). A reference sign 36Fa is a first arm member where a first wire fixing part 51Fa to which the first bending wire 25a is to be fixed is provided. A reference sign 36Fb is a second arm member where a second wire fixing part 51Fb to which the second bending wire 25b is to be fixed is provided. A reference sign 36Fc is a third arm member where a third wire fixing part 51Fc to which the third bending wire 25c is to be fixed is provided.

Each of the plurality of arm members (36Fa, 36Fb, 36Fc) is formed by an elongated plate member, a planar shape of which is substantially rectangular. Here, lengths of the arm members (36Fa, 36Fb, 36Fc) may be a same length or may be mutually different lengths.

The plurality of arm members (36Fa, 36Fb, 36Fc) are fixed with respective proximal ends overlapped with one another. The respective proximal ends of the arm members (36Fa, 36Fb, 36Fc) are fixed by joining the plate members by an adhesive, for example.

Furthermore, the arm members (36Fa, 36Fb, 36Fc) are spread at mutually different angles in a circumferential direction. A distal end of the coupling shaft 50 is connected to a proximal end of each arm member (36Fa, 36Fb, 36Fc).

Furthermore, wire fixing parts (51Fa, 51Fb, 51Fc) are provided near respective distal ends of the plurality of arm members (36Fa, 36Fb, 36Fc). The proximal ends of the corresponding bending wires (25a, 25b, 25c) are fixed to the wire fixing parts (51Fa, 51Fb, 51Fc), respectively.

Here, in the case where the lengths of the arm members (36Fa, 36Fb, 36Fc) are set to be the same, the respective wire fixing parts (51Fa, 51Fb, 51Fc) are at an equal distance from the center axis Ax of the coupling shaft 50 (the bending operation unit 31F).

In the case where the lengths of the plurality of arm members (36Fa, 36Fb, 36Fc) are set to mutually different lengths, a position (distance) of each wire fixing part (51Fa, 51Fb, 51Fc) from the center axis Ax of the coupling shaft 50 (the bending operation unit 31F) may be freely set according to the length of the arm member.

Furthermore, the plurality of arm members (36Fa, 36Fb, 36Fc) are disposed overlapping one another in a direction along the center axis Ax of the coupling shaft 50 (the bending operation unit 31F). Accordingly, distances of the wire fixing parts (51Fa, 51Fb, 51Fc) from the center point P of the rotator 35 in a direction along the center axis Ax of the bending operation unit 31F are set to be mutually different.

In other words, in the example shown in FIG. 34, a reference sign D1 indicates a distance from the center point P of the rotator 35 to the first wire fixing part 51Fa of the first arm member 36Fa in the direction along the center axis Ax. A reference sign D2 indicates a distance from the center point P of the rotator 35 to the second wire fixing part 51Fb of the second arm member 36Fb in the direction along the center axis Ax. A reference sign D3 indicates a distance from the center point P of the rotator 35 to the third wire fixing part 51Fc of the third arm member 36Fc in the direction along the center axis Ax. Here, the distances are set such that D3>D2>D1 is true.

According to the eleventh modification having the configuration as described above, distances from the center point P of the rotator 35 to the wire fixing parts (51Fa, 51Fb, 51Fc) in the direction along the center axis Ax of the bending operation unit 31F can be made different. Accordingly, when pulling the plurality of bending wires (25a, 25b, 25c), pulling can be performed in a more balanced manner. Such a configuration enables pulling to be performed with an ideal amount of wire pulling.

### [Twelfth Modification]

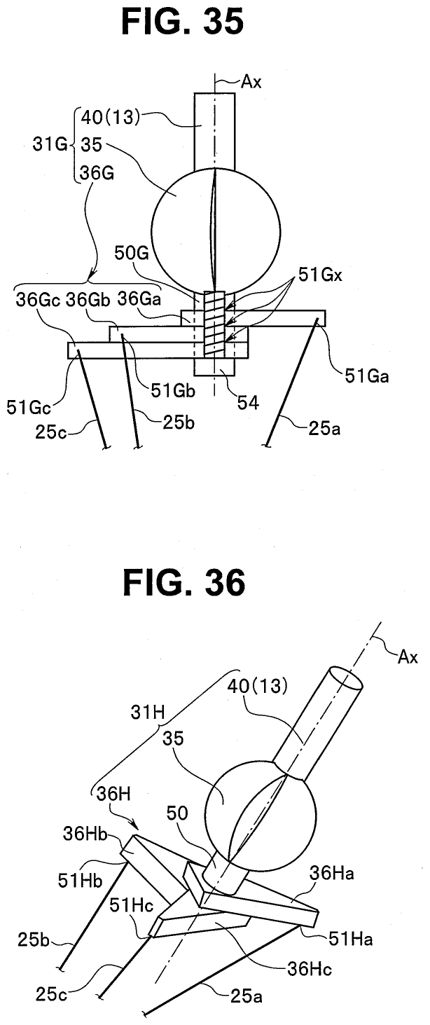

FIG. 35 is a diagram showing a twelfth modification of the endoscope of the embodiment of the present disclosure. More specifically, the twelfth modification is a modification related to the wire fixing member of the bending operation mechanism included in the endoscope of the embodiment of the present disclosure.

As shown in FIG. 35, a wire fixing member 36G of the twelfth modification basically has substantially the same configuration as the configuration in the eleventh modification described above. The wire fixing member 36G of the twelfth modification is different in that a through hole 51Gx is provided on a proximal end side of each of a plurality of arm members (36Ga, 36Gb, 36Gc).

Furthermore, with the wire fixing member 36G of the twelfth modification, a coupling shaft 50G is formed into a substantially cylindrical shape. There is a difference in that an internal thread portion is formed on an inner peripheral surface of the cylinder of the coupling shaft 50G.

In the twelfth modification, there is included a fixing bolt 54 that is inserted through each through hole 51Gx of the plurality of arm members (36Ga, 36Gb, 36Gc), and that is disposed in a direction along a center axis Ax of a bending operation unit 31G with an axial center coinciding with the center axis Ax. The fixing bolt 54 is screwed with the internal thread portion of the coupling shaft 50G after being inserted through each through hole 51Gx in a state where proximal ends of the plurality of arm members (36Ga, 36Gb, 36Gc) are overlapped with one another.

With the bending operation unit 31G of the twelfth modification configured in the above manner, angles of the plurality of arm members (36Ga, 36Gb, 36Gc) relative to one another in a circumferential direction can be freely adjusted.

Furthermore, a plurality of types with mutually different lengths may be prepared in advance as the plurality of arm members (36Ga, 36Gb, 36Gc). Here, when arm members with mutually different lengths are freely selected and used, positions of wire fixing parts defined by the lengths of the arm members in a radial direction around the center axis Ax can be freely selected and set.

According to the twelfth modification having such a configuration, the position of the wire fixing part in the circumferential direction can be very easily changed and adjusted. Furthermore, the position of the wire fixing part in the radial direction around the center axis Ax can be very easily changed.

### [Thirteenth Modification]

FIG. 36 is a diagram showing a thirteenth modification of the endoscope of the embodiment of the present disclosure. More specifically, the thirteenth modification is a modification related to the wire fixing member of the bending operation mechanism included in the endoscope of the embodiment of the present disclosure.

As shown in FIG. 36, a wire fixing member 36H of a bending operation unit 31H of the thirteenth modification basically has substantially the same configuration as the configuration in the eleventh modification described above (FIGS. 33 and 34). With the wire fixing member 36H of the thirteenth modification, a shape of a plurality of arm members (36Ha, 36Hb, 36Hc) is slightly different.

In the thirteenth modification, the plurality of arm members (36Ha, 36Hb, 36Hc) are different in that the plurality of arm members (36Ha, 36Hb, 36Hc) are each made from a plate member, a planar shape of which is substantially triangular. The arm members (36Ha, 36Hb, 36Hc) are fixed with respective proximal ends overlapped with one another. Here, as fixing means for each arm member, fixing means indicated in the eleventh modification (for example, adhesion of plate members) or fixing means indicated in the twelfth modification (for example, fastening using a fixing bolt or the like) is adopted, for example. A plurality of wire fixing parts (51Ha, 51Hb, 51Hc) are provided at (near) vertices of respective arm members (36Ha, 36Hb, 36Hc).

According to the thirteenth modification having the configuration as described above, because the planar shape of the plurality of arm members (36Ha, 36Hb, 36Hc) is substantially triangular, proximal ends can be formed to have a wide width, and strength can be secured, and also, because distal ends are tapered, a weight of the wire fixing member 36H can be reduced.

### [Fourteenth Modification]

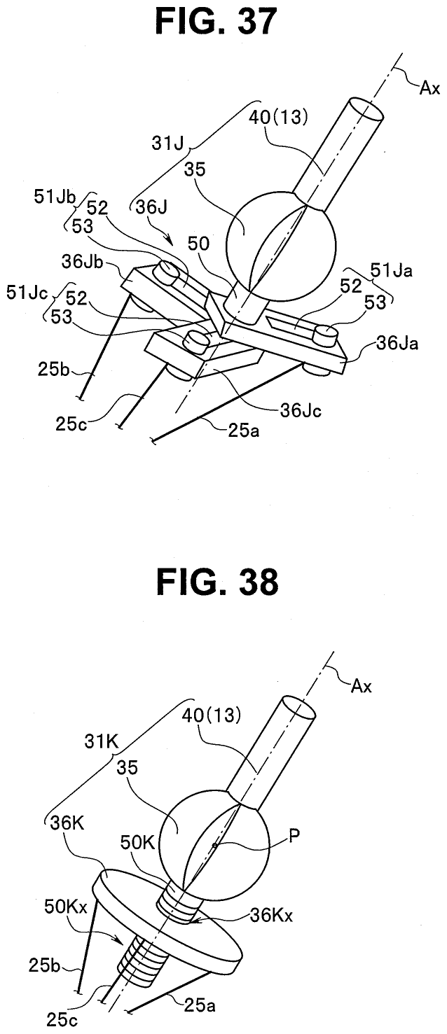

FIG. 37 is a diagram showing a fourteenth modification of the endoscope of the embodiment of the present disclosure. More specifically, the fourteenth modification is a modification related to the wire fixing member of the bending operation mechanism included in the endoscope of the embodiment of the present disclosure.

As shown in FIG. 37, a wire fixing member 36J of a bending operation unit 31J of the fourteenth modification basically has substantially the same configuration as the configuration in the eleventh modification described above (FIGS. 33 and 34). The wire fixing member 36J of the fourteenth modification is slightly different with respect to a mode of a plurality of arm members (36Ja, 36Jb, 36Jc). The arm members (36Ja, 36Jb, 36Jc) include wire fixing parts (51Ja, 51Jb, 51Jc), respectively, the wire fixing parts (51Ja, 51Jb, 51Jc) being configured substantially in the same manner as in the tenth modification described above (FIGS. 31 and 32). The plurality of wire fixing parts here are a first wire fixing part 51Ja, a second wire fixing part 51Jb, and a third wire fixing part 51Jc.

Each wire fixing part (51Ja, 51Jb, 51Jc) includes the long hole 52 for position adjustment and the bolt/nut 53 for fixing. The long hole 52 for position adjustment and the bolt/nut 53 for fixing are configured in the same manner as in the tenth modification.

The fourteenth modification configured in the above manner may achieve a same effect as the eleventh modification. Moreover, as in the tenth modification, fixed position of the bending wires (25a, 25b, 25c) fixed on the arm members (36Ja, 36Jb, 36Jc) of the wire fixing member 36J may be freely set in the radial direction from the center axis Ax.

### [Fifteenth Modification]

FIG. 38 is a diagram showing a fifteenth modification of the endoscope of the embodiment of the present disclosure. More specifically, the fifteenth modification is a modification related to the wire fixing member of the bending operation mechanism included in the endoscope of the embodiment of the present disclosure.

As shown in FIG. 38, a bending operation unit 31K of the fifteenth modification basically has substantially the same configuration as the configuration in the embodiment described above. The bending operation unit 31K of the fifteenth modification is slightly different with respect to modes of a coupling shaft 50K and a wire fixing member 36K.

In the fifteenth modification, the coupling shaft 50K is a shaft member including a thread portion 50Kx (for example, an external thread) on a circumferential surface. A through hole 36Kx is formed in a corresponding manner at a center of the wire fixing member 36K. A thread portion (for example, an internal thread; not shown) where the thread portion 50Kx of the coupling shaft 50K is to be screwed is formed on an inner circumferential surface of the through hole 36Kx.

Note that when the coupling shaft 50K is screwed and assembled with the through hole 36Kx of the wire fixing member 36K, a center of the through hole 36Kx coincides with a center axis Ax of the bending operation unit 31K.

According to the fifteenth modification having the configuration as described above, the coupling shaft 50K and the wire fixing member 36K are coupled by screwing, and thus, a position of the wire fixing member 36K in a direction along the center axis Ax can be easily adjusted.

In other words, a distance between the center point P of the rotator 35 and an arrangement surface of a wire fixing part (51Ka, 51Kb, 51Kc) that is an attachment part for the bending wire (25a, 25b, 25c) on the wire fixing member 36K can be easily adjusted. Therefore, the position of the wire fixing member 36K of the fifteenth modification in the direction along the center axis Ax can be easily adjusted. Accordingly, the present configuration allows the amount of wire pulling to be more appropriately adjusted.

The present disclosure is not limited to the embodiments described above, and various changes and applications may, of course, be made within the scope of the gist of the disclosure. Furthermore, the embodiments described above include disclosures at various stages, and various disclosures may be extracted by combining a plurality of disclosed structural conditions as appropriate. For example, in the case where a problem to be solved by the disclosure can be solved and advantageous effects of the disclosure can be obtained even when some of the structural conditions are excluded from all the structural conditions indicated in the embodiment described above, a configuration excluding such structural conditions may be extracted as a disclosure. Moreover, structural elements of different embodiments may be combined as appropriate. The disclosure is not limited by any specific embodiment, and is limited only by the appended claims.

The above detailed description includes references to the accompanying drawings, which form a part of the detailed description. The drawings show, by way of illustration, specific embodiments that may be practiced. These embodiments are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments may be used, such as by one of ordinary skill in the art upon reviewing the above description. The Abstract is to allow the reader to quickly ascertain the nature of the technical disclosure and is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the embodiments should be determined with reference to the appended claims, along with the full scope of equivalents to which such claims are entitled.

Example 1. An insertion device comprising:
an operation section;
a bending portion provided at an insertion section provided continuously to the operation section;
first to third wires each having a distal end attached to the bending portion, where, when a proximal end is pulled, each wire causes the bending portion to bend in a desired direction;
a lever including a shaft member that extends from inside of the operation section to outside of the operation section, the lever being tilted under an operation force from the outside of the operation section;
a wire fixing member configured to allow each of the proximal ends of the first to third wires to be fixed at mutually different positions inside the operation section, and to pull the first to third wires in conjunction with tilting of the lever; and
first to third wire guides configured to guide the first to third wires, respectively, at mutually different positions in a state where tension is applied, and to separately change each of wire pulling directions, wherein
each of the proximal ends of the first to third wires is fixed to the wire fixing member while being disposed in mutually different quadrants on an XY-plane that is a plane set by an X-axis orthogonal to the shaft member and a Y-axis orthogonal to the X-axis, the XY-plane having a point of origin at an intersection point of the X-axis, the Y-axis, and the shaft member,
the first wire guide guides the first wire to which tension is applied, at a position, on the XY-plane, opposite to the proximal end of the first wire across the X-axis,
the second wire guide guides the second wire to which tension is applied, at a position, on the XY-plane, opposite to the proximal end of the second wire across the X-axis and the Y-axis, and
the third wire guide guides the third wire to which tension is applied, at a position, on the XY-plane, opposite to the proximal end of the third wire across the X-axis and the Y-axis.

Example 2. The insertion device according to Example 1, wherein at least one of the first to third wire guides protrudes from an inner wall of a housing of the operation section.

Example 3. The insertion device according to Example 1, wherein at least one of the first to third wire guides is integrally formed with a housing of the operation section.

Example 4. The insertion device according to Example 1, wherein at least one of the first to third wire guides includes a wire reception portion for receiving the first to third wires.

Example 5. The insertion device according to Example 4, wherein the wire reception portion is a groove provided on the first to third wire guides.

Example 6. The insertion device according to Example 4, wherein the wire reception portion is a low friction member provided on the first to third wire guides.

Example 7. The insertion device according to Example 6, wherein the wire reception portion is formed from fluororesin.

Example 8. The insertion device according to Example 4, wherein the wire reception portion is a pulley.

Example 9. The insertion device according to Example 1, wherein at least one of the first to third wire guides includes
a ball including a through hole configured to allow insertion of the first to third wires, and
a support portion configured to rotatably support the ball.

Example 10. The insertion device according to Example 1, wherein, when a sphere that has, at a center, a fulcrum when the lever is tilted under the operation force and that takes, as a radius, a distance between the fulcrum and the proximal end of at least one of the first to third wires is drawn, the first to third wire guides are disposed outside the sphere.

Example 11. The insertion device according to Example 1, wherein the X-axis is an axis in a direction in which the lever is tilted to bend the bending portion in a left-right direction.

Example 12. The insertion device according to Example 1, wherein the Y-axis is an axis in a direction in which the lever is tilted to bend the bending portion in an up-down direction.

Example 13. The insertion device according to Example 1, wherein the insertion device is an endoscope.

What is claimed is:

1. An operation section for use with an endoscope, comprising:
a shaft;
a wire fixing surface connected to the shaft;
a first wire, a second wire, and a third wire, each of the first through third wires having a fixing part attached to the wire fixing surface; and
a first wire guide configured to guide the first wire, a second wire guide configured to guide the second wire, and a third wire guide configured to guide the third wire,
wherein the first wire includes a first portion between the fixing part of the first wire and the first wire guide,
wherein the second wire includes a second portion between the fixing part of the second wire and the second wire guide, and
wherein in a view from a neutral axis of the shaft, the first and second portions cross.

2. The operation section according to claim 1, wherein the third wire includes a third portion between the fixing part of the third wire and the third wire guide, in the view from the neutral axis of the shaft, the third portion crosses the first portion and second portion.

3. The operation section according to claim 1, wherein the first wire includes a first contact point contacting with the first wire guide,
wherein the second wire includes a second contact point contacting with the second wire guide,
wherein the third wire includes a third contact point contacting with the third wire guide, and
wherein in the view from the neutral axis of the shaft, the first to the third contact points don't overlap each other.

4. The operation section according to claim 3, wherein in a view from a direction perpendicular to the neutral axis of the shaft, the first to the third contact points don't overlap each other.

5. The operation section according to claim 4, wherein the first to the third wire guides extend along the direction perpendicular to the neutral axis of the shaft.

6. The operation section according to claim 1, further comprising:
a fourth wire guide configured to guide the first to the third wires, the fourth wire guide extending along the direction perpendicular to the neutral axis of the shaft.

7. The operation section according to claim 1, wherein when the shaft is tilted relative to the neutral axis, one of the first to the third wire is not loosened.

8. The operation section according to claim 2, wherein in the view from the neutral axis of the shaft, the fixing part of the first wire is located between the second portion of the second wire and the third portion of the third wire.

9. The operation section according to claim 8, wherein in the view from the neutral axis of the shaft, the first portion of the first wire is located between the fixing part of the second wire and the fixing part of the third wire.

10. The operation section according to claim 1, wherein the first to the third wire guides are provided on an opposite side of the shaft with respect to the wire fixing surface.

11. An insertion device, comprising:
an operation section;
an insertion section having a proximal end connected to the operation section, the insertion section including a bending portion at a distal end section of the insertion section;
a lever included in the operation section, the lever including a shaft member that extends from inside of the operation section to outside of the operation section and the lever tiltable under an operation force applied to an outside end of the shaft;
a wire fixing member connected to an inside end of the shaft;
a plurality of wires including a first wire, a second wire, and a third wire, each of the plurality of wires having a distal end attached to the bending portion and a proximal end attached to the wire fixing member; and
a plurality of wire guides included in the operation section, the plurality of wire guides including a first wire guide configured to guide the first wire and apply a tension to the first wire to bend the bending portion in a first bending direction, a second wire guide configured to guide the second wire and apply a tension to the second wire to bend the bending portion in a second bending direction, and a third wire guide configured to guide the third wire and apply a tension to the third wire to bend the bending portion in a third bending direction,
wherein an X-Y plane is defined by an X-axis orthogonal to the shaft and a Y-axis orthogonal to the X-axis, the X-Y-plane having a point of origin at an intersection point of the X-axis, the Y-axis, and the shaft,
wherein the proximal end of the first wire, the proximal end of the second wire, and the proximal end of the third wire are attached to the wire fixing member such that each of the proximal end of first wire, the proximal end of the second wire, and the proximal end of the third wire are located in mutually different quadrants on the X-Y-plane,
wherein the proximal end of the first wire is attached to the wire fixing member at a first position in the XY-plane and, when the first wire is in tension, the first wire guide is at a position, in a view along a normal to the XY-plane, across the X-axis from the proximal end of the first wire,
wherein the proximal end of the second wire is attached to the wire fixing member at a second position in the XY-plane and, when the second wire is in tension, the second wire guide is at a position, in the view along the normal to the XY-plane, across the X-axis from the proximal end of the second wire and across the Y-axis from the proximal end of the second wire, and
wherein the proximal end of the third wire is attached to the wire fixing member at a third position in the XY-plane and, when the third wire is in tension, the third wire guide is at a position, in the view along the normal to the XY-plane, across the X-axis from the proximal end of the third wire and across the Y-axis from the proximal end of the third wire.

12. The insertion device according to claim 11, wherein the operation section includes a housing, and wherein at least one of the plurality of wire guides protrudes from an inner wall of the housing.

13. The insertion device according to claim 11, wherein the operation section includes a housing, and wherein at least one of the plurality of wire guides is integrally formed with the housing of the operation section.

14. The insertion device according to claim 11, wherein at least one of the plurality of wire guides includes a wire reception portion, and wherein the wire reception portion is configured to receive a respective one of the plurality of wires.

15. The insertion device according to claim 14, wherein the wire reception portion is one of a groove, a low friction member, or a pulley.

16. The insertion device according to claim 14, wherein the wire reception portion is formed from a fluororesin.

17. The insertion device according to claim 11, wherein at least one of the plurality of wire guides includes:
a convex surface portion located between a first support portion and a second support portion,
wherein the convex surface portion includes a through hole configured for insertion of one of the plurality of wires, and
wherein the first support portion and the second support portion are configured to rotatably support the at least one of the plurality of wire guides.

18. The insertion device according to claim 11, wherein, when the lever is tilted about a fulcrum under an operation force, the wire fixing member moves along an arc on a surface of an imaginary sphere,
wherein a distance from the fulcrum to the arc defines a radius of the imaginary sphere, and
wherein the plurality of wire guides is located outside the imaginary sphere.

19. The insertion device according to claim 11, wherein, when the lever is tilted in a direction of the X-axis, the bending portion bends in a direction orthogonal to a longitudinal axis of the bending portion, and
wherein, when the lever is tilted in a direction of the Y-axis, the bending portion bends in a direction parallel to a longitudinal axis of the bending portion.

20. The insertion device according to claim 11, wherein the insertion device is an endoscope.
